(19) United States
(12) Patent Application Publication
(10) Pub. No.: US 2025/0062068 A1
(43) Pub. Date: Feb. 20, 2025

(54) ELECTRIC COMPONENTS INCLUDING COILS AND METHODS TO FABRICATE THE SAME BY 3D PRINTING

(71) Applicant: BIOSENSE WEBSTER (ISRAEL) LTD., Yokneam (IL)

(72) Inventors: Nathanel Yehuda David Hayat, Petah Tikva (IL); Shemer Shmaryau Berkowitz, Binyamina (IL); Barry Neal Breen, Givat Zeev (IL); Meir Bar-Tal, Haifa (IL); Daniel Zohar, Zichron Yakov (IL); Avraham Cohen, Kiryat Bialik (IL)

(21) Appl. No.: 18/807,192

(22) Filed: Aug. 16, 2024

Related U.S. Application Data
(60) Provisional application No. 63/533,258, filed on Aug. 17, 2023.

(51) Int. Cl. H01F 41/04; H01F 5/04; H01F 5/06; H01F 17/00; H01F 41/12

(52) U.S. Cl. CPC H01F 41/043; H01F 5/04; H01F 5/06; H01F 17/0013; H01F 41/122

(57) ABSTRACT

Electric components including coils and methods to fabricate the same by 3D-printing are disclosed. The method includes: 3D printing a magnetic material to form a magnetic channel comprising a magnetic core of the coil device; 3D printing a conductive material to form a conductive channel, including conductive windings of the coil device with turns surrounding the magnetic core; and 3D printing a non-magnetic electrically insulating material to form electrical insulation between the turns of the conductive windings of the coil device. In some embodiments functional structures of the electric component, including the turns of the conductive windings of the coil and the electrical insulation between the turns, are each printed with minimal in-layer feature size of at least two voxels of the 3D-printing. Some embodiments facilitate 3D-printing of flattened coil devices having low aspect ratio between their lengths along their magnetic axes and their widths perpendicular thereto.

### CROSS-REFERENCE TO RELATED APPLICATION

This application claims the benefit of U.S. Patent Provisional Application 63/533,258, filed Aug. 17, 2023, the contents of which are incorporated by reference as if set forth in its entirety herein.

### TECHNOLOGICAL FIELD

The present invention is in the field of magnetic field sensors and specifically relates to techniques of fabricating electric components including coils which are suitable for magnetic-field-based position sensing, particularly for sensing the positions of medical devices.

### BACKGROUND

In various therapeutic and diagnostic procedures, a catheter/probe is inserted into a patient's body (e.g., chamber of the heart) and to be brought into contact with a body tissue there. Typically, in such procedures, it is necessary to determine the catheter's location within the body (i.e., the location at which a distal tip of the catheter engages the body tissue) as well as the pressure applied thereby to the tissue.

Catheters having integrated location and pressure sensors for sensing the location of the catheter and the pressure/force applied thereby at the contact region with the tissue, are generally known. Such catheters typically utilize inductive coils for determining the location of the catheter within the body and/or the pressure/force applied thereby to a body tissue it engages with.

Conventional techniques for such catheters often utilize wire-winded coils to which a ferrite core may be manually inserted after the winding. The coils fabricated in this way are then soldered to a cable that carries the coil signal to processing circuits, and fitted within the catheter's body.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A and 1B illustrate a 3D-printed electric component 100 including a coil 120, according to an embodiment of the invention;

FIG. 1C illustrates a cross section of the coil in the electric component 100 of FIGS. 1A and 1B;

FIG. 2 is a flow diagram of a method 200 to fabricate, by 3D-printing, of one or more coils 120, or electric components 100 including the same, according to some embodiments of the present invention;

FIG. 3A illustrates a perspective view of the electric component 100 including a coil 120, according to another embodiment of the present invention;

FIG. 3B illustrates a side view of a coil 120 within the electric component 100, according to yet another embodiment of the present invention;

FIG. 3C illustrates a top view of a cross section of the coil 120 within the electric component 100 of FIGS. 3A and 3B;

FIG. 3D illustrates the cross section of the coil 120 shown in FIG. 1C with grid lines indicating the distribution of pixels/voxels withing a certain 3D-printed plan/layer of the coil 120;

FIG. 3E illustrates a perspective cross-section view of the conductive windings 124 of the coil 120, taken along the cross-section plane CP shown in FIG. 3D;

FIGS. 3F and 3G illustrate two respective section cuts of the conductive windings 124 taken along the respective section planes SP1 and SP2 shown in FIG. 3E;

FIGS. 3H and 3I are two respective side views of the conductive windings 124, taken respectively from lateral side A of the coil 120 and from lateral side C of the coil 120 of FIG. 3A.

FIG. 4A illustrates a perspective view of a 3D printed electronic component 100 which includes/formed-as a flattened coil 120 having low/small aspect ratio between its length along its magnetic axis and its width, according to yet another embodiment of the present invention;

FIG. 4B illustrates a side view of the flattened coil 120 3D printed in the electronic component 100 of FIG. 4A;

FIGS. 5A, 5B, 5C and 5D illustrate several views of an electric component, in these examples a coil device 120, with helical magnetic core, 3D printed according to some embodiments of the present invention;

FIGS. 6A, 6B and 6C illustrate several views of an electric component, in these examples a coil 120 with a plurality magnetic subchannels, each including a helical shaped magnetic core section, which is 3D printed according to some embodiments of the present invention;

FIGS. 7A and 7B illustrate several views of an electric component, in these examples a coil 120 with meander-like shaped magnetic core, 3D according to some embodiments of the present invention.

FIGS. 8A and 8B illustrate several views of an electric component, in these examples a coil 120 with coiled magnetic core, 3D according to some embodiments of the present invention.

FIG. 9 is an illustration of a medical instrument 1000 furnished having a magnetic-field based positioning sensor 300 including coils configured according to an embodiment of the present invention.

Like reference numerals are used in the figures to designate elements/parts of the electric components of the invention which have similar functionalities. For brevity and conciseness, the description of such similar elements/parts is not necessarily repeated with respect to each of the embodiments, yet it should be appreciated that in embodiments where not otherwise indicated, the description of elements/parts having the same configuration and/or functionality made in any one of the embodiments, may also be applicable in other embodiments.

### DETAILED DESCRIPTION OF EMBODIMENTS

There is a need in the art of catheters, for physically agile and sensitive catheters having compact/narrow dimensions, that are capable of providing accurate feedback on the location of the catheter in the body, and the contact interface between the catheter and the tissue and the force (e.g., vector) applied between them.

U.S. patent No. U.S. Pat. No. 8,535,308 and U.S. patent application No. U.S. 2020/015693, which are both assigned to the assignee of the present application, disclose for example configurations of such catheters utilizing sensory circuits having several coils, for sensing the location and orientation of the catheter body, as well as the force applied thereby at the interface with the tissue.

One challenge in the art of such catheters is to fit accurate location and force sensors within the narrow body of the catheter, whose lateral dimensions are typically only several millimeters. Indeed, as indicated above, in conventional catheters technologies, the location and force sensors generally utilize miniature wire-winded coils to which a ferrite core may be manually inserted with an open magnetic circuit configuration in order to yield sufficient induced-voltages suitable for location and force sensing of the catheter components. These conventional techniques are however time consuming and costly and involve delicate manual operations for fitting of the coils within the small dimensions of the catheters body.

Therefore, there is a need in the art for more efficient, cost-effective techniques for fabrication of such catheters and for the fabrication of small high sensitivity coils (i.e., having high induced voltage in response to magnetic fields) suitable for use for location and or force sensing in catheters such as those disclosed above. Moreover, there is a need in the art for automated fabrication techniques of such coils, or of electronic components comprising the same, that is suitable for mass production with yield of standardized coils having small variability between fabricated coils of the same design. Further, in the interest of further advent in the art of catheters, there is also a need in the art to facilitate fabrication of even smaller and/or of higher impedance/induced-voltage, coils so as to improve either or both of the sensitivity and agility, and/or reduced dimensions, of the catheter.

The present invention provides a novel and inventive electric component including one or more coils suitable for use as/in magnetic field sensor of a magnetic-field-based positioning system. Moreover, the present invention provides a novel and inventive method of fabrication of the electric components including such coil(s) utilizing 3D printing technique. In embodiments the electric component may be fabricated with coil(s) of compact dimensions while providing relatively high induced-voltage in response to sensed magnetic fields, and is therefore particularly suited for use as a position sensor for sensing the positions of compact medical device, such as catheters having narrow dimensions.

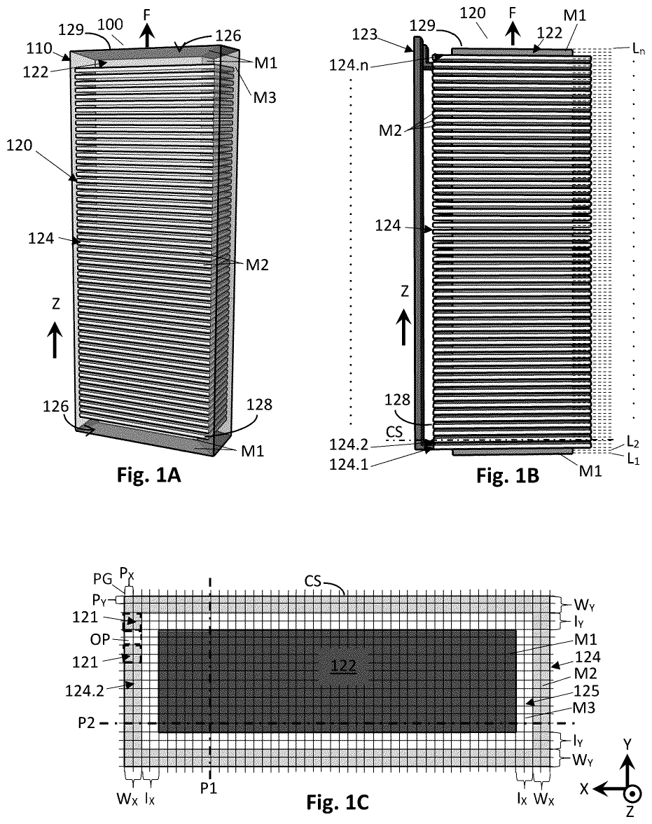

With reference made together to FIGS. 1A and 1B an example of an electric component 100 including a coil 120, according to an embodiment of the invention, is illustrated. The component 100 includes a body 110 with a at least one coil 120 defined/fabricated therein by multi-material 3D printing.

According to the technique of the invention the electric component 100 (and the coil(s) 120 therein) is fabricated by 3D printing of s of at least three different 3D-printable materials M1 to M3 including:
At least one type of magnetic material M1 which is used/3D-printed to form at least a magnetic channel(s) 129 of the coil 120, which typically includes magnetic core 122 of the coil 120. Optionally, the magnetic channel 129 also includes magnetic flux collectors 126 which may be coupled to the magnetic core 122 from opposite ends thereof and have larger surface/cross-section area than the core facilitating channeling of higher magnetic flux through the core to thereby improve the coil's sensitivity to magnetic fields from a desired magnetic field direction.
At least one type of conductive (generally non-magnetic) material M2 which is used/3D-printed to form a conductive channel 128 of the coils 120 including conductive windings 124 and contact terminals 123 thereof (the contact terminals are not specifically shown in FIG. 1A); and
At least one type of dielectric/non-electrically-conductive material M3 which is used/3D-printed to form a bulk of the body 110 of the electric component 100 at regions from which the magnetic and conductive materials are excluded.

Spacings between adjacent windings/turns 124 are occupied by 3D printed non-electrically-conductive material. The non-electrically-conductive material may for instance be the non-magnetic dielectric material M3 which is 3D printed in the bulk of the body 110 of the electric component 100 and/or the magnetic material M1 used for printing the magnetic channels in cases where it is non-electrically conductive (i.e. sufficiently electrically insulative material).

FIG. 1A illustrates a perspective view of the electric component 100 with the coil 120 therein, and with the bulk material M3 of the body 110 shown/exemplified in translucent color/shade so that the coil 120 therein is visible. FIG. 1B illustrates a side view of a part of the coil 120 which is 3D printed within the electric component 100 shown in FIG. 1A, while excluding (not showing) the bulk material M3 of the body 110 of the electric component 100 and also excluding (not showing) the optional flux collectors 126 in order to illustrate the coil 120 with better clarity.

As illustrated the coil 120 includes the magnetic channel 129, which includes in this example the core 122 of the coil 120 with two optional magnetic flux collectors 126 arranged from opposite ends thereof. The coil 120 also includes an electric channel 128 arranged/configured therein to yield induced voltage through the electric terminals 123 (not specifically shown in FIG. 1A) of the electric channel 128 in response to magnetic flux propagation through the core 122.

To this end, it should be understood that in various embodiments the magnetic channel 129 and the electric channel 128 may have different shapes. Generally, at least one of the magnetic 129 and electric 128 channels has coiled/twisted geometry (e.g. a helical or otherwise twisted geometry) and is wrapped/surrounded or otherwise twisted about the other one of the magnetic 129 and electric 128 channels, in order to yield the induced voltage in the electric channel 128 in response to magnetic flux propagation through the core 122 of the magnetic channel 129.

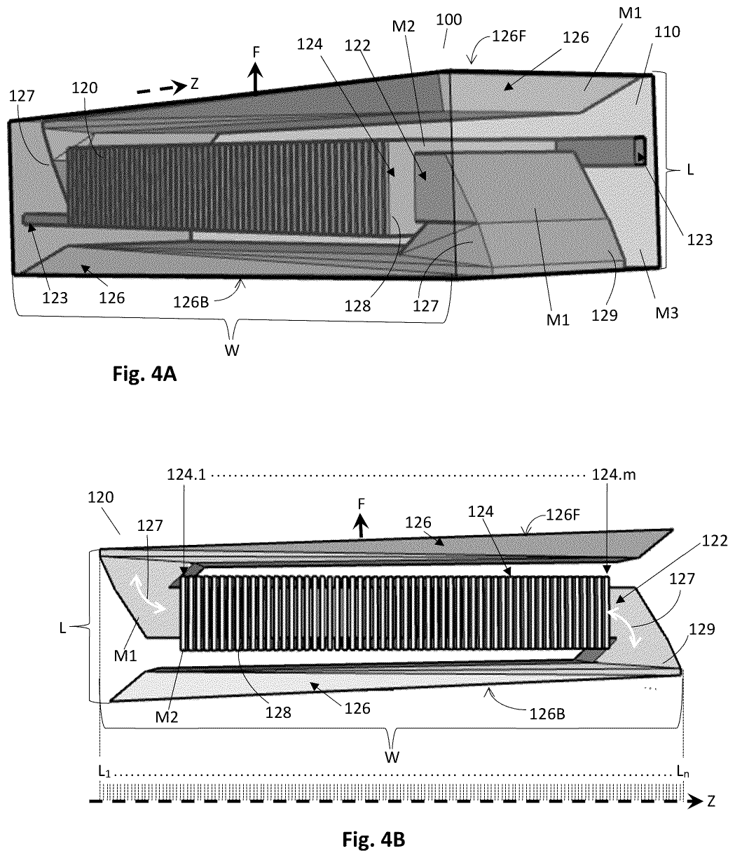

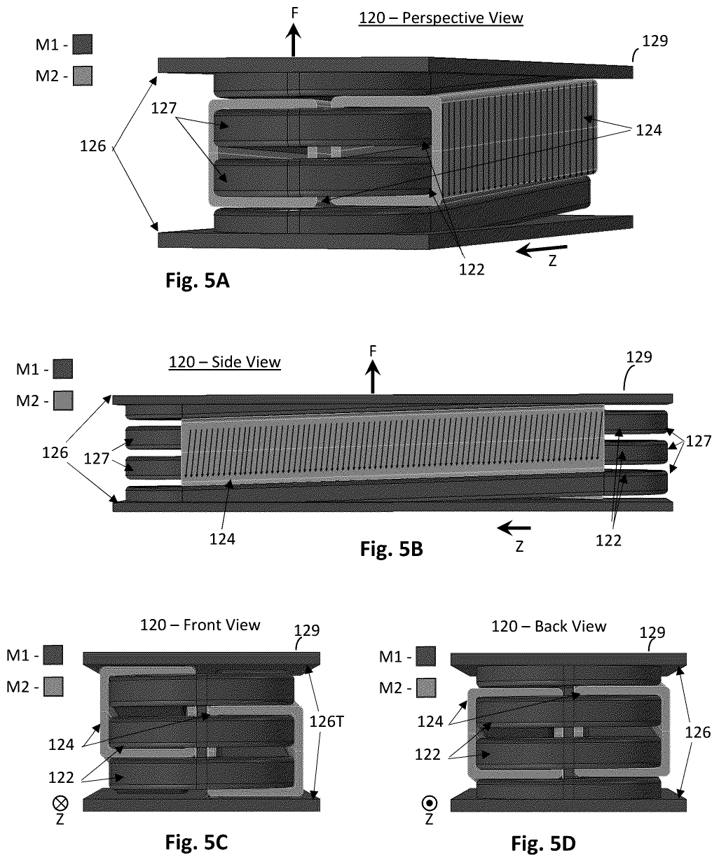

In this regard, in the non-limiting example of FIGS. 1A and 1B, a magnetic core 122 having a straight/rod-like shape is exemplified in the magnetic channel 129, while the electric channel 128 has conductive windings 124 turned/twisted/coiled such that it form a plurality of turns 124.1 to 124.n wrapped/surrounded about the magnetic core 122 of the magnetic channel 129, so that induced voltage is produced in the electric channel 128 in response to magnetic flux through the core 122 of the magnetic channel 129. In this connection it should be noted that the magnetic channel 129 and/or the magnetic core 122 thereof are not necessarily shaped to form straight/rod-like core and may be 3D printed with other shapes that are turned/twisted/coiled within the body of the electric component 100. For instance, the core 122 may be formed with helical, meander-like or coiled shape with the windings 124 surrounding multiple regions thereof. Such core shapes may be used in order to facilitate generation of sufficiently high induced voltage of the coil 120 desired sensitivity of the coil 120 to magnetic fields from a desired magnetic field direction/axis F, while with desired dimensional constraints of the coil 120 or of the electric component 100 in its entirety. For instance, in FIGS. 4A and 4B described in more details below, an example of a 3D printed coil 120 having low/small aspect ratio between its length along its magnetic axis F and its width/lateral dimension (which is in this case the direction Z of the 3D printing) and; in this example the magnetic channel 129 of the coil 120 has a twisted shape and turns 127 of the channel 129 between the flux collectors 126 and the core 122 such that the core 122 extends along the longer, lateral, dimension of the coil 120 and has a greater length to accommodate conductive windings 124 surrounding it, than would have being in case of a straight core extending along the shorter length of the coil 120 along magnetic field axis F. In another examples, shown for instance in FIGS. 5A to 8B described in more details below, various 3D printed coils 120 according to embodiments of the invention are illustrated with their magnetic channels 129 (e.g. magnetic cores 122) having twisted shapes of either a helical, multi/double helical, meander, and/or coiled, shapes; whereby these twisted core shapes facilitate a greater length of the core 122 passing through windings of the electric channel 128 or otherwise surrounding the electric channel 128 thereby increasing the amount of magnetic flux inducing voltage in the electric channel 128 to improve the sensitivity of the coils while facilitating various desired dimensional constraints thereof (to enable fitting them within small medical devices).

Optionally, as also exemplified in FIG. 1A, the magnetic channel 129 of the coil 120 includes flux collector(s) 126 magnetically coupled-to the core 122 of the channel 129. The flux collector(s) 126, typically a pair of them coupled to opposite sides of the core 122, are configured with respective relatively large facets/surfaces perpendicular to the magnetic axis F of the coil 120 (e.g. typically larger than the cross-section area of the core 122) such that they can collect magnetic flux propagating along the direction of the coil's magnetic axis F, and channel the flux through the core (thereby improving the sensitivity of the coil). As indicated the flux collectors are optional, and are included in the magnetic channel 129 in some embodiments where the cross-section of the magnetic core 122 is small/narrow and/or when the magnetic core 122 does not extend in direction substantially parallel to the magnetic axis F.

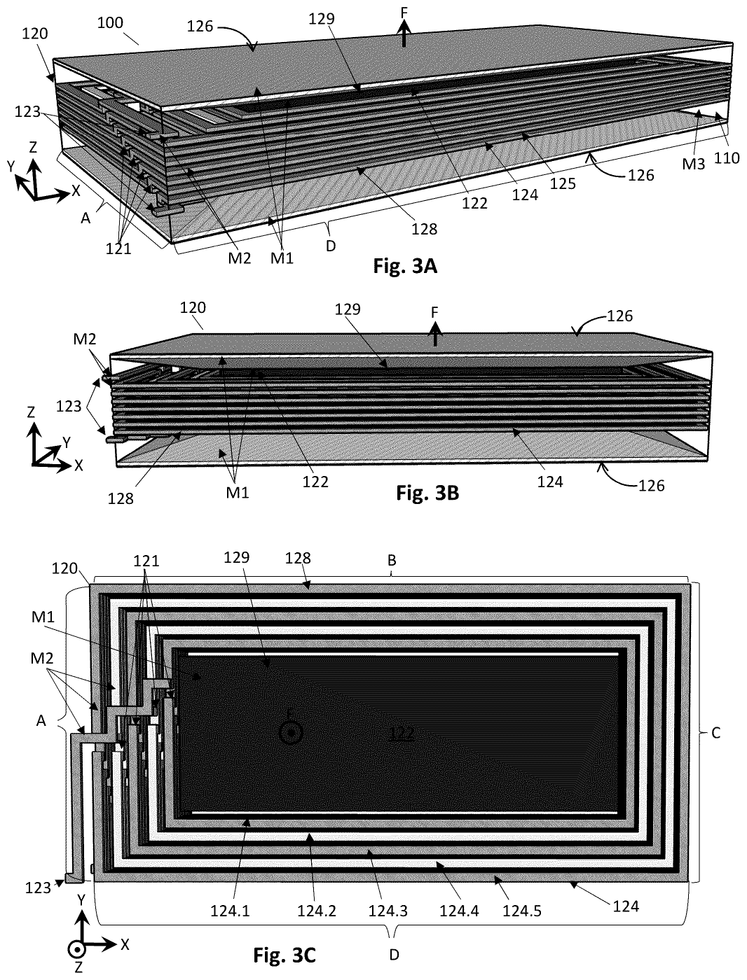

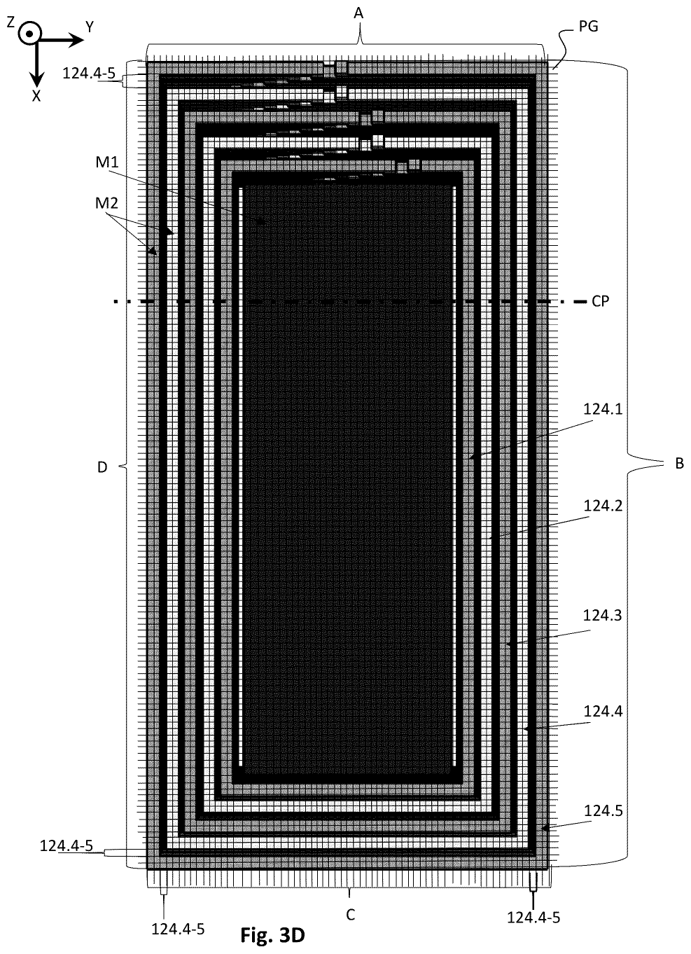

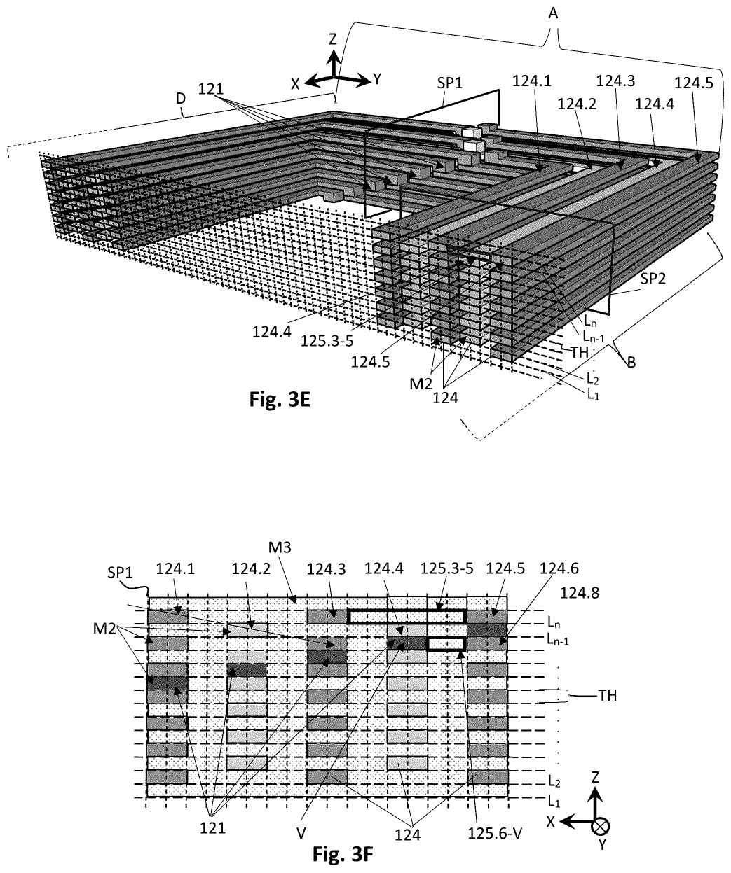

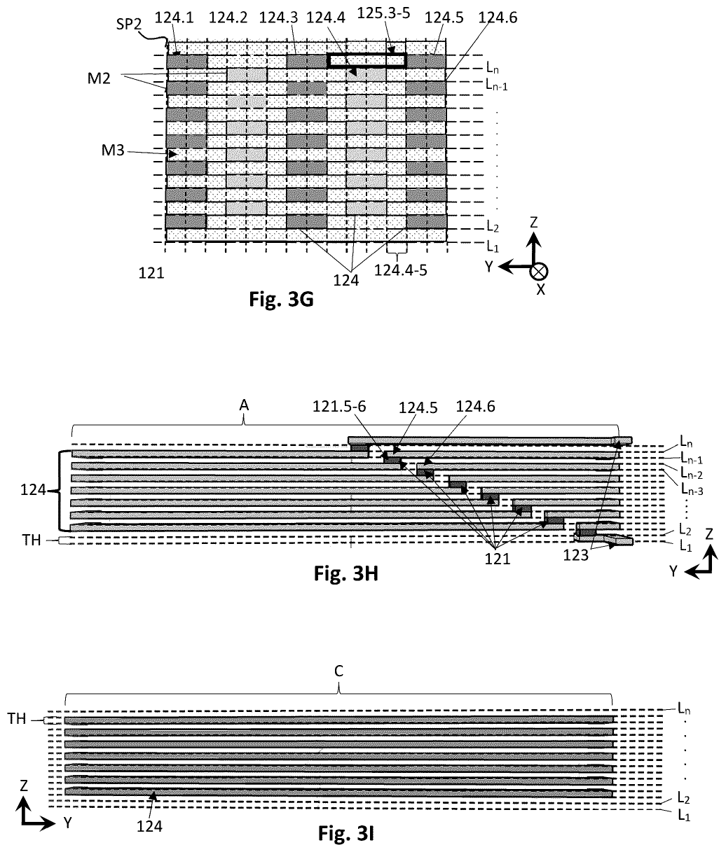

Optionally, as also exemplified in FIG. 1A, the electric channel 128 of the coil 120 includes conductive windings 124, including a plurality of turns surrounding the magnetic core 122, in order for voltage to be induced therein in response to magnetic flux through the core 120. The conductive windings 124, are optional, as in some implementations, as indicated above, the magnetic core 120 may be itself winded/turned to surround the electric channel 128 such that magnetic flux propagating therein circumferences the electric channel 128 and induces voltage therein. In various embodiments, the conductive windings 124 may be configured/3D-printed to surround the magnetic core 120 in a coiled shape/path (as illustrated in the example of FIG. 1A) and/or with spiral shape/path within one or more of the printed layers, and/or with the shape of multiple concentric coils (as illustrated in the example of FIGS. 3A to 3I).

Generally, as would be appreciated by those versed in the art, the electric component 100 of the present invention may include/be-fabricated-with one or with a plurality of coils 120 therein, as well as optionally with other electric circuit elements (not specifically illustrated) such as capacitors and/or resistors and/or electrical connections between them. A person of ordinary skill in the art will readily appreciate how to fabricate/3D-print such other electric circuit elements within the electric component 100 of the invention, while optionally utilizing additional 3D printable materials not specifically exemplified herein, such as electrically resistive material for fabrication of resistors, and or material of selected dielectric properties to be 3D printed in between capacitor's conductive plates.

According to some aspects of the present invention, the electric component 100 of the invention is fabricated utilizing 3D printing method (additive manufacturing technology) that includes a layer-by-layer deposition (and optionally curing), along a printing direction Z, of the at least three printable materials M1 to M3 indicated above. Typically the at least 3 types of printable materials include: the magnetic material(s) M1, which is used for printing the magnetic channels 129 of the coils 120 (including the magnetic cores 122 and the optional flux collectors 126 thereof); the conductive material(s) M2 which is used for printing the electric channels 128 of the coils (including the conductive windings 124 and the electric contacts/terminals thereof); and non-magnetic electrically insulating (dielectric) material, which is 3D printed to form the bulk of the body 110 of the electric component 100, as well as typically/optionally to fill the spacings between the windings 124 of the coils to electrically insulated between them.

The terms magnetic-material and/or particles is used herein to designate materials of high relative magnetic permeability ur, for example having relative permeability preferably of μᵣ≥100 relative to the vacuum permeability μ₀, and more preferably in some cases in the order of μᵣ≥500, or even in the order of μᵣ≥5000. Conversely the terms non-magnetic-material and or particles are used herein to designate materials of low relative permeability μᵣ which is substantially lower than that of the magnetic materials used in the electronic components' fabrication and typically in the order of μᵣ˜1.

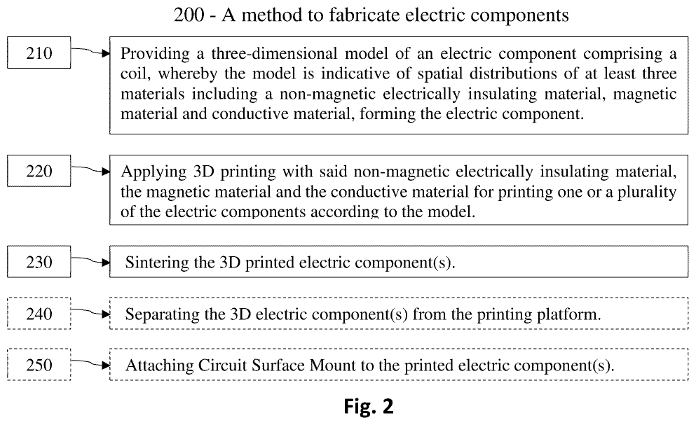

With reference to FIG. 2, a flow diagram of a method 200 to fabricate the electric component 100 according to some embodiments of the present invention by multi-material 3D-printing is illustrated. The multi-material 3D-printing in this case utilizes at least three different materials M1 to M3 for printing the electric component(s) 100.

In operation 210 of method 200, a model of one or more electric component 100 according to embodiments of the present invention are provided. The model may include one or more electric component 100 configured with the principles of the invention as described above with reference to FIGS. 1A and 1B and/or as described below with reference to FIGS. 3A to 8B. The model is indicative 3D spatial distribution (voxel maps) of the at least three materials, M1 to M3, which are to be 3D-printed to fabricate the electric component(s) 100 of the present invention. Particularly the model is indicative of the respective spatial distributions of the three materials M1 to M3 that should be 3D printed to form an electric component 100 with at least one coil 120 according to the invention.

Typically, the multi-material 3D-printing according to method 200 operates by successive printing of layers, one on top the other, along the printing direction Z (see for instance the layers indicated L₁, L₂ to Lₙ in FIGS. 1B, 3D, 3E, 3H and 3I). The spatial distributions of the three materials M1 to M3 in the model is such that the printing results with one or more electric components 100 each including at least one coil 120 configured according to an embodiment of the present invention.

In operation 220 of the method 200, a multi-material 3D-printing is applied to print the one or more magnetic field sensors 100 based on the spatial distributions of the three or more materials M1 to M3 in the model. The multi-material 3D-printing may be carried out for example by utilizing a photopolymerization 3D-printing technique to print layers L₁, L₂ to Lₙ that are composed of a combination of several materials having different electromagnetic properties (e.g., two three or optionally more than three materials with different electromagnetic properties may be printed in each layer). In a typical example, 3D printing of the electric component 100 described above involves printing of spatial distributions of the at least three materials M1 to M3 having the different electromagnetic properties.

To this end in various embodiments the electrically conductive material M2 may include for example Silver and/or Copper and/or Palladium, and/or Silver-Palladium alloy. The magnetic material M1 may include for example Ceramic-Ferrite material and/or Metal Material. In some embodiments the magnetic material M1 is a soft magnetic material having relative permeability μᵣ of at least 100 or above. Preferably the magnetic material M1, particularly that used for printing magnetic channel 129 having coiled/twisted or otherwise non-straight shapes, is a material having relative permeability μᵣ in the order of 500 or above, and even more preferably in the order of 5000 or above, in order to facilitate efficient channeling of magnetic flux through the folds/curves 127 thereof. The non-magnetic dielectric (electrically insulating) material M3 (also referred to herein as the bulk material) may for instance include ceramic and/or glass-ceramic.

The 3D printing of the electric component(s) 100 may be implemented for example based on the principles of Vat Photopolymerization, utilizing for instance stereolithography (SLA) or digital light processing (DLP) for the pattern implementation/projections. The invention is not limited to these specific technologies and may generally be implemented by other multi-material 3D printing technique as may be developed to be suitable for the fabrication of the electric components according to the invention.

With the technique of the invention the electric component(s) 100 with small feature sizes can be printed utilizing 3D printed layers with layer-thicknesses in the scale down to microns (e.g., sintered layer thickness may be about 8-10 microns). The pixelated in-layer resolution of the printing may be with pitch/widths of the printed voxels in the order of about 12-25 microns laterally within the layers (i.e. with respect to the lateral coordinates X, Y).

In some embodiments of the invention functional features of the electric component(s) 100 are printed with minimal in-layer feature size (of at least two voxels, in order to avoid malfunctions of the electric component(s) 100 due to misprints of single/individual voxels. Accordingly, features of the electric component(s) 100 which are printed within a layer may be printed with characteristic in-layer lateral sizes as small as about 24-50 microns or less (this is considering that the lateral pitch/widths of the voxels within the layer is of about 12-25 microns and considering the preference to print each important/functional feature with minimal in-layer feature size of at least two the voxels).

In this regard, reference is made to FIG. 1C showing a cross section of the electric component(s) 100 shown in FIG. 1B along the plane/printed-layer CS marked there, which passes across the winding turn 124.2. As shown the turn 124.2 is not closed (opened at region OP) such that it is connected, via conductive vias 121 to other turns of the conductive windings 124 above and below it (in this manner forming the conductive windings 124 in the coil's electric channel 128). It should be noted that the term via (e.g. referring to elements marked 121 in the figures) is used herein to designate parts/portions of the conductive channel 128 (e.g. integral parts thereof) which are 3D printed to electrically connect between turns of the conductive windings 124 that are printed in above or below one another in different layers.

FIG. 1C shows a pixels' grid PG illustrating the voxel distribution within a certain printed layer of the electric component 100, with P_{X} and P_{Y} marking the voxels' lateral width/pitch along each of the lateral (in-layer) X and Y dimensions (which, as said, may be in the order of 12-25 microns when printing compact electric components). As illustrated, in this non-limiting example, the electric channel 128 including the turns of the windings 124 of the coil 120 (which are a functional element thereof) are printed with minimal in-layer feature size of at least two voxels. Also, insulation 125 separating parts of the electric channel 128 from other parts thereof (e.g. such as between turns 124.1 to 124.n) of the windings 124, or from other optionally conductive elements of the electric component (such as the core 122 which may be conductive in this non-limiting example), are printed with minimal in-layer feature size of at least two voxels.

In this connection the phrase minimal in-layer feature size is used herein to design the minimal lateral size in voxels/pixels, of a functional feature of the electric component along any lateral (X and/or Y) directions of the 3D-printing within each layer in which it is being printed. This means that in this example, by “cutting” any part of a functional feature, such as the electric channel 128 and/or the insulation 125, across any printed layer with orientation of the cut plane perpendicular to the printing direction (i.e. parallel to the layer), the visible dimensions of the cross-section area of the functional feature within the cut (within the layer) would be not less than two 3D-printed voxels/voxels, which are filled (or supposed to be filled in case of a misprint) with the suitable material (e.g. M2 for the conductive channel 128 and M3 for the insulation 125) along each of the X and Y lateral dimensions/axes. In other words, the minimal in-layer cross-section area of any functional feature is at least 2×2 voxels/pixels printed within the layer. In this regard it is noted that in at least some of the 3D printing techniques, features of size of a single pixel/voxel within the printed layer (e.g. single pixels/voxels isolated from any sequence of consecutive pixel/voxels of the same material within the printed layer), may occasionally be misprinted. Accordingly, by ensuring that the functional features of the electric component 100 of the invention are printed with the minimal in-layer features size of at least two voxels along each lateral direction X and Y, provides for mitigating such misprints of single voxels, and ensures that even if a single voxel is misprinted within the layer, the respective functional feature in which the misprint occurred will still operate properly. With respect to the insulation 125, it should be noted that in various embodiments, where the insulation 125 is 3D printed with the same material as the bulk of the monolithic body 110, there may not be any specific features to distinguish the insulation 125 from other parts of the monolithic body 110. Therefore it should be clarified that the term insulation 125, is used herein considering its functional property, to electrically insulate between to conductive features of the electrical component 100, such as between winding turns, and while considering this functional property, any set of voxels of electrically insulative material which are printed within each layer along the shortest path(s) between to conductive features in the layer, should be considered as a part of the insulation 125 between the said conductive features. Accordingly, with this definition, the minimal in layer feature size of the insulation is such that the shortest insulative path(s), in voxels, between conductive features/voxels within a layer extend through at least two voxels of the layer.

Accordingly, any individual misprint of single voxel in the electric channel 128 will not cause electrical disconnection in the electric channel 128, and any individual misprint of single voxel in the insulation 125 will not affect electric shortcut in the electric channel 128. In like manner it should be understood that also the magnetic channel 129 is printed with the minimal in-layer features size of the at least two voxels (in some implementations it is 3D printed with in-layer features sizes substantially larger than two voxels in order to facilitate efficient propagation of magnetic flux therethrough).

To this end, in this example the turns of the electric channel 128 is printed with in-layer lateral widths W_{X} and/or W_{Y} of at least two voxels along at least one of the lateral dimensions X and Y, and with height of one layer so that they have at least said minimal in-layer feature size of the two voxels. Accordingly, any individual voxel misprint in the winding will not cause electrical disconnection in the electric channel 128 and will not disrupt the operation of the coil 120. Additionally, as in this specific non-limiting example magnetic material M1 of which the core 122 is made is electrically conductive metal (i.e. note that as described below the core 122 and/or the magnetic channel 129 are not necessarily made of conductive metal—see the discussion below regarding the printing of the magnetic channel 129 with electrically insulating ceramic ferrite material), and therefore insulation 125 is printed between the conductive windings 124 and the core 122 to prevent shortcut of the windings 124. The insulation 125 is also printed with respective in-layer lateral widths I_{X} and I_{Y} of at least two pixels along the lateral dimensions X and Y, such that its minimal in-layer feature size is at least two voxels. Accordingly, any single voxel misprint in the insulation 125 will not cause shortcut in the electric channel 128 and will not disrupt the operation of the coil 120. To this end, the method 200 facilitates robust and reliable fabrication of the electric components, by utilizing printing each functional feature of the electric component 100 with in-layer feature size of at least two voxels.

For carrying out the 3D printing, curable resins corresponding respectively to the at least three materials M1, M2 and M3 are provided. More specifically the curable resins used in the 3D printing of the magnetic field sensor 100 of the invention include for example the following:
A magnetic material resin which includes particles of a soft magnetic material magnetic material M1 suspended in curable polymer binder. The particles of the magnetic material M1 may include for example particles of Ceramic-Ferrite material. It should be noted that alternatively in some embodiments the particles of the magnetic material M1 may include particles of magnetic Metal Materials such as Iron alloy, iron-silicon alloy, iron-aluminum-silicon alloys, low-carbon steels, nickel-iron alloys, iron-cobalt alloys, ferrites, amorphous alloys, as well as in some cases Nickel-Cobalt Alloy (particularly in cases it has soft magnetic qualities). In embodiments where miniature coils are fabricated within the monolithic body 110 the particles of magnetic material used may be provided for example with characteristic sizes in the micron to submicron scale, particularly in cases where the magnetic channels 129 of the coils manifest delicate features.
A conductive material resin which includes particles of conductive material M2 suspended in curable polymer binder. The particles of the conductive material M2 may for instance include Silver material, Copper material, Palladium material, and/or Silver-Palladium alloy. As specific choice of suitable conductive material may generally depend on the required temperatures of the sintering/firing process required to finalize the printing of the electric components and may thus depend on the types of magnetic and dielectric materials. In embodiments where miniature coils are fabricated within the monolithic body 110, the particles of conductive material M2 used in the printing may have characteristic sizes in the submicron scale and as small as in the nanometer scale.
A dielectric and non-magnetic material resin which includes particles of non-magnetic dielectric material M3 suspended in curable polymer binder. The particles of the non-magnetic dielectric material typically include ceramic or glass-ceramic particles which can withstand the high sintering/firing temperatures required for sintering the printed magnetic and the conductive materials, M1 and M2. Also, here the size of the particles may be selected according to the minimal feature sizes that should be occupied by the dielectric material/bulk of the magnetic field sensor, and for fabrication of miniature coils may be selected for instance in the order of nanometer to submicron scale.

In regards to the material selection in the above, it should be noted that although printing magnetic channel 129 with magnetic metal material (e.g. electrically conductive) is generally possible, in various embodiments printing the magnetic channel with an electrically insulating ceramic ferrite may be preferred. One reason for that is that the use ceramic ferrite facilitates achieving more compact arrangement of magnetic and electric channels, 129 and 128. This is because soft magnetic metal materials (non-ceramic ferrite) that are suitable for use for the magnetic channel printing, are typically conductive. Therefore, a magnetic channel 129 printed with a non-ceramic ferrite, should be spaced from the electric channel 128, at least at some places, in order to prevent short circuit, which might impact the achievable miniaturization of the magnetic field sensor 100 and/or require more complex design thereof. Therefore, in some embodiments preferably ceramic ferrite material is used for the printing of the magnetic channels 129.

Thus, utilizing the at least three curable resins described above, 3D printing of the magnetic field sensor 100 may be performed layer by layer along a printing direction Z utilizing for example photopolymerization multilateral 3D printing techniques such as SLA or DLP. The printing direction Z is typically selected such that it is substantially perpendicular to planes spanned by the turns of the conductive windings 124 of the coils 120. To this end, as indicated above, the choice of the printing direction Z may have particular importance in the printing of miniature magnetic field sensors 100 because the lateral feature resolution of the printing in the X-Y plane is lower than the “vertical” resolution of the layers obtained along the printing direction Z.

To this end, typically the 3D printing of each layer of the sensor 100 typically includes the following (not necessarily in that order):
Deposition/printing and curing the magnetic material M1 at regions of the layer corresponding to the magnetic channels 129 of the coils 120;
Deposition/printing and curing the conductive material M2 at regions of the layer corresponding to electric channel 128 of the coils 120; and
Deposition/printing and curing the non-electrically-conductive (dielectric) non-magnetic material M3 to form the bulk of the monolithic body 110 of the sensor 100.

It should be noted that in some embodiments, the 3D printing is conducted such that the conductive material M3 of the coils 123 is fully embedded within the bulk of electric component's body 110 in order to protect the conductive material M3 against degradation (e.g. oxidation) due to environmental conditions, particularly during the high temperature sintering 230 which follows layer by layer material deposition and curing.

Thus, by the consecutive printing and curing of successive layers in operation 220. according to the spatial distribution of the materials in the sensor's model, one or more 3D structures of one or more sensors 100 each including at least one longitudinally oriented coil and at least one transversely oriented coil electric coil 220 are obtained.

Method 200 further includes operation 230 for sintering the one or more 3D structures of the one or more electric components 100 printed in operation 120. The sintering is generally performed at temperature sufficient for burning or driving off polymers (binding polymers) from the one or more magnetic field sensors 100 as well as firing ceramic or glass ceramic materials such as M3 and optionally M1 which are used in the printing. To this end, after the sintering the material density of the printed ceramic and metal materials (e.g., M1, M2 and M3) of the electric components 100 approach 100%. For example, in embodiments where the conductive material M2 mainly includes Silver, the sintering may be performed at temperatures below the Silver melting point (e.g., at temperatures not exceeding 961° C.; preferable in this case the sintering temperature may be limited up to about 900 C in order to achieve a stable sintering process). In other embodiments where the conductive material includes a Silver-Palladium alloy, the sintering may be performed at temperatures below the alloy melting point (the specific melting point depends on the ratio of silver and palladium in the alloy and depending on the composition the melting point is generally between the melting point of silver, 961° C., and that of palladium, 1,555° C.). In another example, in embodiments where the conductive material M2 includes Copper, the sintering may be performed in an Oxygen deprived environment (so as to avoid oxidation of the Copper), and at temperatures below the 1084° C. Copper melting point.

In this connection, it should be noted that in some embodiments of the present invention the method 200 is carried out for 3D-printing electric components 100 in which miniature coils having features of micron scale size are embedded. For instance, the 3D printed layers may be printed with thicknesses in a range of about 5 to 15 μm (e.g., 9 μm), depending on the specific model design and 3D-Printing technology used. Accordingly, a pitch of the turns of the windings 124, which may be in the order of twice the pitch of the 3D printed layers, may be within the range of about 10 μm to 30 μm along the printing direction Z of the 3D printed layers (e.g., typically turns' pitch of about 18 μm).

Further, optionally (depending on the specific printing technology used), the method 200 may include operation 240 for separating printed electric components 100 from the building/3D-printing platform (e.g., 201 shown in FIG. 1E). Generally, depending on the type of building platform (e.g., and whether it can withstand the sintering 230), the operation 240 may be performed before or after the sintering 230. Indeed, in some embodiments of the invention, particularly where miniature/delicate electric components are printed, which may be damaged by carrying out their separation from the building platform before the sintering, a suitable building platform may be used which can withstand the temperatures of the sintering 230, and the electric components' separation from the platform may be conducted post sintering so as to avoid/reduce damage to the sensors during the separation process.

In view of the above, method 200 of the present invention provides a novel and inventive technique for fabrication of electric components 100 each including one or more coils 120 as described above. The method 200 is suited for fabrication of miniature electric components 100 (with features sizes as small as few microns) that are operable as/in a magnetic-field-based positioning sensors and suited for use in compact/narrow medical devices such as catheters. The method 200 may be used in mass production to simultaneously 3D-print large pluralities of miniature such electric components 100 built on the same platform (e.g., typically between hundreds and several thousand electric components 100 may be printed using for instance a vat photopolymerization technology). Each electric component 100 may have for example a ceramic/glass-ceramic body 110 embedding therein one or more highly sensitive open magnetic circuit coils 120. The coils 120 may be fabricated with small dimensions, for example in the order of ˜0.5 mm to ˜2 mm and may provide high sensitivity/induced-voltage in response to magnetic fields, for example in the range ˜0.1 to 1 μV/(Gauss*Hz).

Reference is now made to FIGS. 3A to 3I illustrating a 3D printed electric component 100 including at least one coil 120 configured according to an embodiment of the present invention. The electronic component 100 includes a magnetic channel 129 and a conductive channel 128 of the coil 120. The magnetic channel 129 and conductive 128 channels are 3D printed with magnetic material M1 and electrically conductive material M2 respectively. The magnetic channel 129 includes a magnetic core 122 of the coil 120, and the conductive channel 128 includes an arrangement of conductive windings 124 with a plurality of turns surrounding the magnetic core 122 (e.g. some of the turns are individually marked with reference numerals 124.1 to 124.6 in the figures). The body/bulk 110 of the electric component 100 is 3D printed with non-magnetic electrically insulating material M3. To this end the electrical insulation 125 between the turns of the windings 124 is in this example 3D printed with the same non-magnetic electrically insulating material M3 used for printing the bulk of the body 110.

In the FIGS. 3A to 3I: FIG. 3A shows a perspective view of the complete electric component 100; FIG. 3B is a side view of the coil 120 within the electric component 100 in which the non-magnetic electrically insulating material M3 is not illustrated for clarity of the figure; FIG. 3C shows a top view of the coil 120 within the electric component 100, with the flux collectors 126 omitted/not-shown for clarity; FIG. 3D shows a top view of the coil 120 without showing the electric terminals 123 and showing a pixel/voxel grid PG illustrating the distribution voxel of the 3D printing as captured from the top side of the coil 120; FIG. 3E illustrates a cross-section of the windings 124 of the coil 120 with the insulation 125 between them across lateral sides B and D of the coil 120 (in which no conductive vias 121 between turns are accommodated), taken along the cross-section plane CP shown in FIG. 3D; FIGS. 3F and 3G illustrate section cuts taken along the respective section planes SP1 and SP2 of the coil 120 shown in FIG. 3E, showing the windings 124 with the insulation 125 between them respectively across the lateral side A (in which the conductive vias 121 between turns in different layers are accommodated) and across the lateral side C of the coil 120 (in which now conductive vias 121 are accommodated); and FIGS. 3H and 3I are two respective side views showing some of the turns of the conductive windings 124 (without showing the insulation 125 between them), which are taken respectively from lateral side A of the coil 120 along which conductive vias are accommodated between the windings 124 and from lateral side C of the coil 120 along which conductive vias are not accommodated in this example. In these figures, where applicable, axes indicating the longitudinal direction Z (being the layer-by-layer-printing direction of the electric component 100) and the lateral directions X and Y perpendicular thereto, are illustrated. Also, where applicable, guide lines illustrating the 3D printed layers L₁ to Lₙ of the electric component 100, the lateral side A, B, C and D thereof, and the magnetic axis F of the coil 120 therein, are illustrated in the figures.

For example, as illustrated in FIG. 3H, the via 121.5-6 is printed in layer Lₙ₋₂ to connect between the turns 124.5 and 124.6 which are 3D printed in the layers above and below it, Lₙ₋₁ and Lₙ₋₃, respectively.

As indicated above, preferably according to some embodiments of the present invention, functional features of the 3D printed electric component(s) 100 are 3D printed with minimal in-layer feature size of at least two voxels.

In the embodiment of the electric component 100 of FIGS. 3A to 3I, a configuration of the coil's 120 windings 124, which facilitates compact arrangement of large number of windings' turns, while maintaining a minimal in-layer feature size of at least two voxels in the windings' turns 124 and in the electric insulation 125 between them, is illustrated. This configuration thus facilitates the reliable 3D printing of highly sensitive and compact coil(s) in the electric component, since on the one hand any individual voxel misprint in the electric channel 128 or the insulation will not disrupt proper operation of the coil, while on the other hand it provides for stacking large number of windings' turns 124 surrounding the magnetic core 122 thereby facilitating the high sensitivity of the coil 120.

To achieve that one or more of the following arrangement features are implemented in the arrangement of the 3D printed turns 124.1-124.n, and the insulation 125 in the spacings between turns:
a. The conductive channel 128 (including the turns of the conductive windings 124) as well as the electrical insulation 125 between the windings' 124 turns, are each 3D printed with the minimal in-layer feature size of the at least two voxels; This contributes to the reliability of the electric component 100, and or of its production by the 3D printing as discussed above.
b. The turns, e.g. 124.1-124.n or some of them, of the conductive windings 124 are arranged/3D-printed planarly within the printed layers L₁ to Lₙ. Accordingly since the thicknesses TH of the printed layers/voxels (along the printing direction Z) is typically smaller than the lateral X, Y dimensions of the voxels, printing the turns plenary within the layers facilitates stacking greater number of turns.
c. A plurality of concentric turns are printed at least some of the layers L₁ to Lₙ so as to facilitate printing of greater number of turns surrounding the core 122. For instance, in this example turns 124.1, 124.3 and 124.5 are concentric turns printed plenary within layer Lₙ; and turns 124.2 and 124.4 are concentric turns printed plenary within layer Lₙ₋₁.
d. A plurality of concentric turns are printed in consecutive layers so as to facilitate printing/stacking of a greater number of turns surrounding the core 122. For instance, in this example turns 124.4 and 124.5 are concentric turns printed plenary within the consecutive layers Lₙ₋₁ and Lₙ respectively.
e. Concentric turns that lay (3D printed) in consecutive layers and that are concentrically adjacent (in embodiments that such turns exist) are laterally separated, from one or more lateral sides of the concentric, by only one lateral voxel between them. This is in order to facilitate printing/stacking of a greater number of turns surrounding the core 122. For instance, in the present example the turns 124.4 and 124.5 are concentrically adjacent turns laying respectively within the consecutive layers Lₙ₋₁ and Lₙ. The lateral separation 124.4-5 between them (with respect to the X and/or Y axes) is of only one voxel along the lateral sides B, C, and D of the electric component 100 (i.e. along the lateral side(s) at which no conductive vias 121 are accommodated), and of two voxels (e.g. at least two) along the lateral side A (e.g. along the side where the conductive vias are accommodated), as illustrated in FIGS. 3D and 3E. In this regard, it should be noted that the phrase concentrically adjacent turns is used herein to designate turns that are adjacent to one another with respect to the radial direction of the concentric arrangement of turns. In this regard it should be noted that the invention facilitates minimal lateral spacing of only one voxel between turns (along lateral sides not accommodated with vias 121), while maintaining the desired two-voxel minimal in-layer feature size of the insulation 125 between turns. This in turn facilitates compact packing of winding turns high sensitivity of small dimension 3D printed coils. In this regard it is noted that the lateral separation 124.4-5 is printed with single pixel width only from the lateral sides of the concentric (in this example B, C and D) at which there are no vias connecting the concentrically adjacent turns to other/neighboring turns above or below them. Therefore, some embodiments also implement the arrangement feature f., which is described in the following, in order to maximize/optimize the length along the concentrically adjacent turns along which they can be spaced from one another by only one voxel.
f. Conductive vias 121 which connect each of the concentrically adjacent turns to other/neighboring turns above or below them, or to other parts of the conductive channel 128, are preferably arranged from as few lateral sides of the concentric as applicable by the geometry and number of turns of the specific coil (e.g. preferably if applicable from one side only), such that from other one or more sides the concentrically adjacent turns can be laterally spaced from one another by only one voxel while maintaining the minimal in-layer feature size of the insulation 125 of the at least two-voxels between conductive features (e.g. turns and/or vias) in the same layer (as desired for manufacturing reliability of the electric component). In this specific non-limiting example, the vias 121 are all arranged along one lateral side A of the concentric turns, whereby in order to maintain the minimal in-layer feature size (at least two-voxels) of the insulation 125 between the turns along that lateral side A the lateral spacing between concentrically adjacent turns along that side is of two voxels.

To this end, the combinations of the above features a. to f., and in particular the combination of the features d. c. and f. facilitate compact packing of conductive turns while maintaining the minimal in-layer feature sizes of each functional feature of the coil which is desired for printing reliability. FIGS. 3F and 3G, illustrate respective section cut SP1 of the windings 124 across the respective lateral sides A and B thereof in which the conductive vias 121 are respectively accommodated and not accommodated. As shown in FIG. 3G although a lateral separation, such as 124.4-5, between concentrically adjacent turns in different layers, such as between 124.4-124.5, is of only one voxel along the sides where no vias are accommodated, the insulation between the adjacent turns within the same layer, such as the insulation 125.3-5 between 124.3 and 124.5 within the same layer, has a lateral width greater than the minimal in-layer feature size, in this example a widths of four voxels along the side(s) at which no vias are accommodated. As shown in FIG. 3F since the lateral separation, such as 124.4-5, between concentrically adjacent turns in different layers, such as between 124.4-124.5, is of more than one voxel (two in this example) along the sides where vias are accommodated (e.g. side A in this case), the insulation between the adjacent turns within the same layer along these sides, such as the insulation 125.3-5 between 124.3 and 124.5 along the side A, has also a lateral width greater than the minimal in-layer feature size, in this example a widths of six voxels along the side(s) A at which the vias are accommodated. Otherwise, if for example the lateral spacing 124.4-124.5 would have been made with width of one voxel, as it is along sides B to D, the minimal in-layer feature size of the insulation in the spacing/insulation such as 125.6-V between the adjacent turn such as 124.4 and 124.6 within the same layer would have been of only one voxel (e.g. due to the presence there of the via between them (e.g. the via indicated V in the figure which connects between neighboring turns above one another), and would thereby not satisfy the minimal in-layer feature size condition for the insulation 125. Therefore in some embodiments of the invention conductive vias 121 which connect between neighboring turns above or below one another are arranged along as few lateral sides as possible whereby the lateral spacing between concentrically adjacent turns along these sides is of at least two voxels, thereby permitting the lateral spacing between the concentrically adjacent turns along other lateral sides to be of only one voxel to yield one the one hand a compact arrangement of the turns and on the other hand electrical insulation with minimal in-layer feature size of at least to voxels facilitating reliability of the coil's fabrication.

In this example, the electric component 100 is 3D printed by successive printing of a plurality of printed layers L₁, L₂ to Lₙ along the printing direction Z. Each of the turns, e.g. 124.1 to 124.6, of the conductive windings 124 is arranged/3D-printed planarly within one of the printed layers L₁ to Lₙ. For example, as shown in FIGS. 3E to 3I, the turns 124.1, 124.3, and 124.5 are 3D printed planarly within layer Lₙ and the turns 124.2, 124.4 are 3D printed planarly within layer Lₙ₋₁. To this end in this example the turns are printed in the multitude of the printed layers. The magnetic core is printed such that passes through a consecutive multitude of the printed layers L₁ to Lₙ and surrounded by the turns thereat. The electric component 100, i.e. the coil 120 thereof, includes electrical insulation 125 that is 3D printed between the turns of the conductive windings 124. In this non-limiting example the electrical insulation 125 is 3D printed with the same bulk material M3 which is also used to print the body 110 of the electric component 100.

As indicated above, in this example the turns (e.g., 124.1 to 124.6) of the conductive windings 124 and the insulation material 125 between them (as well as the magnetic channel 129) are 3D printed with minimal in-layer feature size of at least two voxels of the 3D printing. In order to obtain the highly sensitive coil, in this example the windings 124 includes concentrically adjacent turns that are 3D printed in consecutive layers of the 3D printing with lateral separation of one voxel between the concentrically adjacent turns (at least along some lateral sides of the concentric). As indicated above, lateral separation of the one voxel between concentrically adjacent turns is from one or more lateral sides of the adjacent concentric turns (in this example from sides B to D), and the conductive vias are 3D printed in at least one other lateral side of the concentric turns (in this example from side A). Along the at least one other lateral side (in this example side A) at which the vias 121 are printed/accommodated, the lateral separation between the concentrically adjacent turns is of at least two voxels, in order to maintain a minimal in-layer feature size of the insulation to be at least two voxels.

Reference is now made together to FIGS. 4A and 4B illustrating an electric component 100 according to another embodiment of the present invention, which is/includes a coil 120 having a flattened shape with respect to its magnetic axis F or in other words a coil 120 having a low/small aspect ratio between its length L along its magnetic axis F and its width W traverse to its magnetic axis F. For brevity a coli with such dimensions is referred to herein as a flattened coil.

Similarly to the electric component 100 described above with reference to FIGS. 1A to 1C, also in this embodiment of FIGS. 4A and 4B, the electric component 100 includes: a body/bulk 110 that is 3D printed with non-magnetic electrically insulating material M3; a magnetic channel 129 that is 3D printed in the body 110 with magnetic material M1 and includes a magnetic core 122 of the coil 120; and a conductive channel 128 that is 3D printed in the body 110 with electrically conductive material M2 and includes conductive windings 124 with a plurality of turns surrounding the core 122. The and conductive channel 128 is terminated at both ends thereof by electric contacts/terminals 123 (not specifically shown in FIG. 4B). FIG. 4A is a perspective view of the electric component 100 and 4B is a side view of the coil electric component 100 in which the bulk material of the body 110 is not shown.

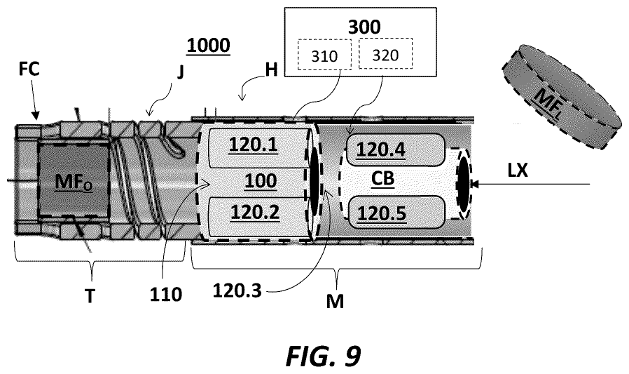

As indicated above, in this embodiment, the required dimensions of the electric component 100 with the coil 120 therein, are such that the electric component 100, and/or the coil 120 therein, is flattened with respect to the direction of its magnetic axis F. In this regard flattened coils, or otherwise coils with such flattened dimensions, are in many cases required for medical devices' position sensing, and in particular are used/fitted in elongated and narrow medical devices, such as catheters, whose positions are often sensed by measuring magnetic field in their surroundings (particularly used for sensing the vector components of the magnetic fields which are perpendicular to the elongated body of the medical device/instrument)—see for instance FIG. 9 and its related description below.

However, one difficulty of 3D printing of such flattened coils with sufficient sensitivity for applications such as position sensing, is that the number of windings 124 turns than can be 3D printed such that they are stacked along the relatively short length L of the coil 120 (i.e. along its dimension parallel to the coli's magnetic axis F). Furthermore, as will be appreciated by those versed in the art, due to their geometry, flattened coils with magnetic core have low apparent permeability (also known as effective permeability) which may be <2, regardless of high permeability of the magnetic material.

Some aspects of the present invention provide for overcoming this difficulty, and 3D printing flattened coils 120 with sufficient sensitivity. This is achieved by 3D printing the magnetic channel 129 such that it includes at least a pair of flux collectors 126 having respective flux collection facets 126F and 126B that are perpendicular to the magnetic axis F of the coil 120. The magnetic channel 129 is 3D printed such that it has curved/folded path with curves/folds 127 defining between them one or more sections of the magnetic core 122 of the magnetic channel 129 which extend along the width dimension W of the coil 120 (i.e. traverse to the coil's magnetic axis F). The magnetic channel is thereby adapted to collect, via facets 126F and 126B, magnetic flux propagating from the direction of the magnetic axis F and channel the magnetic flux, via curves 127 to propagate along the core section(s) 122 thereof which extends along the longer width dimension W of the flattened coil 120. In turn the windings 124 of the conductive channel 128 of the flattened coil 120 include a plurality of turns 124.1 to 124.m that are arranged to surround the core section(s) 122 which extends along the longer width dimension W of the flattened coil 120. Accordingly, the number of turns 124.1 to 124.m that can be arranged in this manner is larger than the number of turns that could be arranged in case the core 122 was to extend in the direction along the magnetic axis F (i.e. along the shorted longitudinal dimension L of the coil 120) and accordingly improved sensitivity of the coil to magnetic field components from the magnetic axis F is obtained, despite the flattened shape of the coil 120 in this embodiment.

In this regard it should be noted that according to some embodiments, the flux collection facets/surfaces 126F and 126B that face the magnetic axis F of the coil 120, are made wider (e.g. with substantially larger area) than the characteristic width (e.g. cross section area) of the magnetic channel 129 between them. Even more specifically the areas of the flux collectors' facets/surfaces 126F and 126B, are preferably made substantially larger than the characteristic cross section area of the magnetic core section(s) 122 which are facing a different, e.g. perpendicular, direction to the magnetic axis F. This is in other in order for the flux collectors 126 to collect much more magnetic field flux from the direction of the magnetic axis F than the amount of flux that would be collected to the magnetic channel 129 from other directions, thus making flattened coil with the turned/folded magnetic channel 129 sensitive to magnetic field components directed along the magnetic axis F, while negligibly sensitive to magnetic field components from other directions. Optionally the flux collectors 126 and/or other sections of the magnetic channel are also tapered towards the magnetic core sections 122 (e.g. to facilitate efficient flux channeling from the relatively wide flux collection facets/surfaces 126F and 126B thereof to the narrower magnetic core 122).

As indicated above, in some embodiments the 3D printing technology used for the electric component's 100 fabrication, provides for printing layers with thicknesses that are substantially smaller than the lateral widths of the printed voxels (in other words: the layer resolution is substantially higher than the resolution of in-layer pixels that can be 3D printed). For instance, in certain non-limiting examples the thicknesses of the printed layers are of about 8-10 μm (along the Z direction; typically 9 μm) while the lateral sizes/widths of the printed voxels within the layers are of about 12-25 μm. Therefore, in such embodiments, 3D printing the turns 124.1 to 124.m of the windings 124 with planar orientation in the printed layers facilitates printing of thinner turns (e.g. of about the layer thickness e.g. of 9-10 μm) and with smaller pitch of the turns, than the turn thicknesses and pitch achievable if the turn are printed with other/transverse orientation relative to the printed layers (e.g. which would result with turn thicknesses of about the lateral voxel's width, e.g. 12-25 μm, in case the turns are printed perpendicularly to and across the layers). Therefore, optionally as illustrated in FIGS. 4A and 4B, in some embodiments the direction Z of printing of the flattened coil 120 and/or the electric component 100 in which it may be contained (the direction along which the 3D printing is carried out layer by layer) is chosen/set such that the turns 124.1 to 124.m are printed planarly within the printed layers L₁ to Lₙ (for instance the printing direction Z of the flattened coil 120 may be set parallel to the longer width W dimension of the flattened coil 120 as illustrated in the figures). This facilitates printing a stack of further more number of turns 124.1 to 124.m surrounding the core section 122 (with higher density of turns) and thus further improving the sensitivity of the coil 120.

It should be noted that other features of the flattened coil 120 that is exemplified in FIGS. 4A and 4B, and which are not specifically described with respect to this example, may have similar properties as those discussed with reference to the example of the coils in FIGS. 1A to 1C and/or with in FIGS. 3A to 3I. For instance, optionally, in some non-limiting embodiments, functional features of the flattened coil 120, including its conductive and magnetic channels, 128 and 129, and/or the different parts thereof, as well as insulation 125 between conductive elements such as the turns 124.1 to 124.m, may be 3D printed with minimal in-layer feature size of at least two voxels of the 3D printing in order to facilitate reliable 3D printing of the coil 120 that is robust to misprints of individual voxels. This may be in like manner as described above with reference to FIGS. 1A to 1C. Also, the turns 124.1 to 124.m of the windings may optionally include pluralities of concentric turns (not specifically shown), in order to further increase the count of turns surrounding the magnetic core 122, and thus improve the coil's sensitivity. In such embodiments, optionally the spacings/insulation 125 between concentric turns may optionally be configured in like manner as described with reference to the embodiment of FIGS. 3A to 3I.

In FIGS. 4A and 4B one optional configuration of the 3D printed flattened coil 120 (and/or of an electric component 100 containing the same) is exemplified. The magnetic channel 129 of that flattened coil 120 being curved/folded 127 such that one or more sections of the magnetic channel 129 extends substantially along the longer width dimension W of the flattened coil (perpendicular to its magnetic axis F) printing direction to serve as the magnetic core 122 of the coil and is being surrounded by conductive turns of the windings 124. FIGS. 5A to 8B provide self-explanatory depictions of several other non-limiting examples of configurations of coils 120 whose magnetic channels 129 are curved/folded in order to facilitate configurations of windings to have a plurality of magnetic core sections 122 facilitating an arrangement of the winding turns and/or increasing their number improving the coils sensitivity. Like reference numerals are used in FIGS. 5A to 8B to designate elements/sections of similar functionality as in the coils described above with reference to FIGS. 1A to 1C and 3A to 4B.

FIGS. 5A to 5D respectively exemplify, in self-explanatory manner, perspective-side-, front- and back-views of an electric component, in this example including a coil 120, which may be 3D printed according to embodiments of the present invention. In this example the magnetic channel 129 is curved/folded 127 several times such that between the flux collectors 126, the magnetic channel 129 is printed-with/has a helical-like shape with a plurality of magnetic core section 122 thereof extending substantially along the printing direction Z. The plurality of core sections 122 are surrounded by turns of the conductive windings 124 of the conductive channel, which are planarly printed in the 3D printed layers, with relatively small pitch/distances between them. The turns of the conductive windings 124 are typically spaced from one another by electrical insulation printed in the layers between them (e.g. the electrical insulation may be printed with between the turns with the bulk material M3). As shown the magnetic channel 129 is formed in a helical like shape, such that the multiple magnetic core sections 122 thereof traverse through the conductive windings 124 to channel magnetic flux, which flows between the flux collectors 126 along the magnetic channel 129, to pass several times through the conductive windings 124 and thereby provide improved sensitivity of the coil 120. Although in this non-limiting example the magnetic channel 129 is folded such that two magnetic core sections 122 thereof traverses through the windings 124, it should be appreciated by those versed in the art that the coil 120 may generally be printed with other configurations of the helical channel and may be formed with any suitable number of folds 127 to yield any suitable number of magnetic core sections through the windings that provide the optimized sensitivity for the specific sensory application for which it is used.

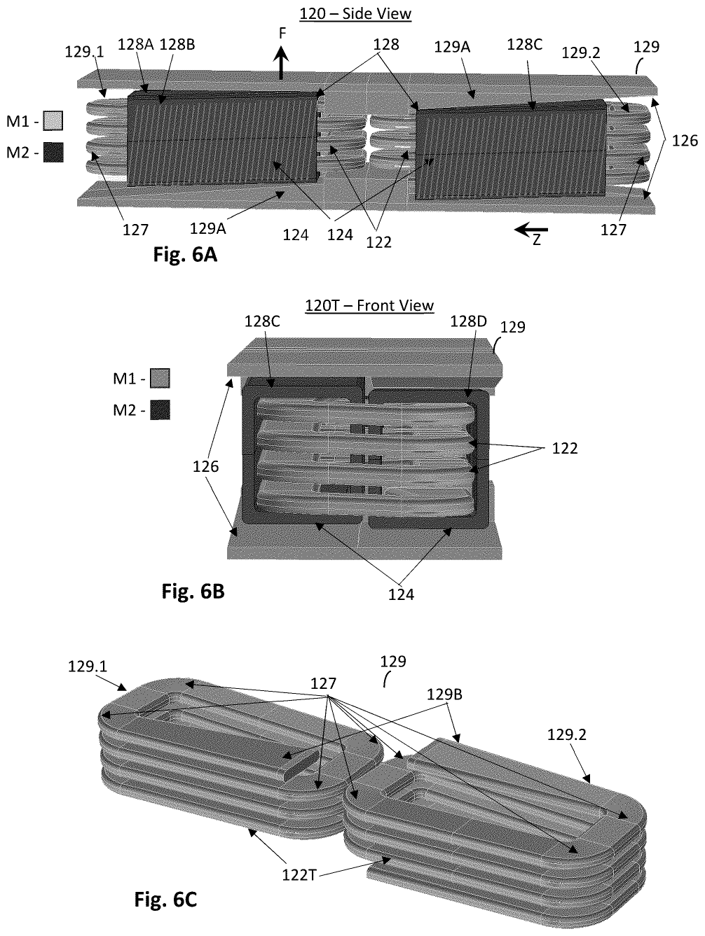

FIGS. 6A and 6C respectively exemplify, in self-explanatory manner, side- and front views of a coil 120 with a plurality of helical magnetic sub-channels, 129.1 and 129.2, which may be printed in the electric components 100 according to embodiments of the present invention. FIG. 6C is a perspective view of the helical magnetic sub-channels In this example, between the flux collectors 126, the magnetic channel 129 is split into a plurality of magnetic subchannels (two in this example 129.1 and 129.2) each being curved/folded 127 several times in the form of a helix (or otherwise a helical-like shape), such that the magnetic channel 129 has a multi-helix shape (double helix in this example—one helix formed by each of the magnetic subchannel 129.1 and 129.2). The magnetic sub-channels 129.1 and 129.2 are printed with curves/folds 127 such that they define a plurality of magnetic core sections 122 extending substantially along to the printing direction Z and are surrounded by turns of the conductive windings 124 of the coil 120. In this non-limiting example, the conductive windings 124 include several winding sets 128A to 128D each including a plurality of conductive turns surrounding different one or more of the magnetic core sections 122. To this end, in various embodiments, the electric channel 128 between the electric contacts/terminals thereof (not specifically shown in these figures), may be 3D printed as a single conductive path passing between the pair of electric contacts/terminals and shaped to form the winding sets 128A to 128D along a single conductive path, or the electric channel 128 between the electric contacts may be printed to split into several (e.g. 4) electric sub channels (e.g. which may be connected to one another near the electric contacts in parallel) and each forming a plurality of windings/turns of the one or more winding sets 128A to 128D.

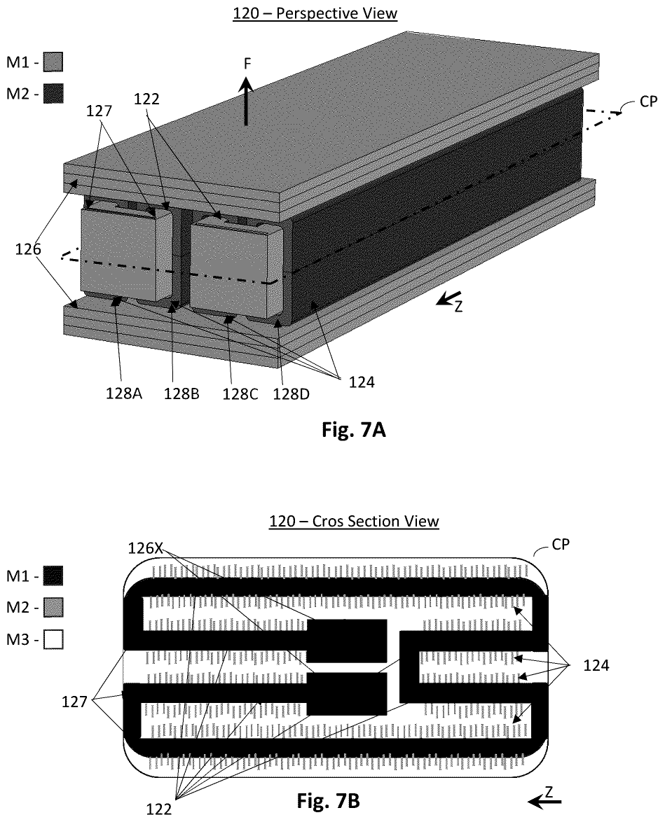

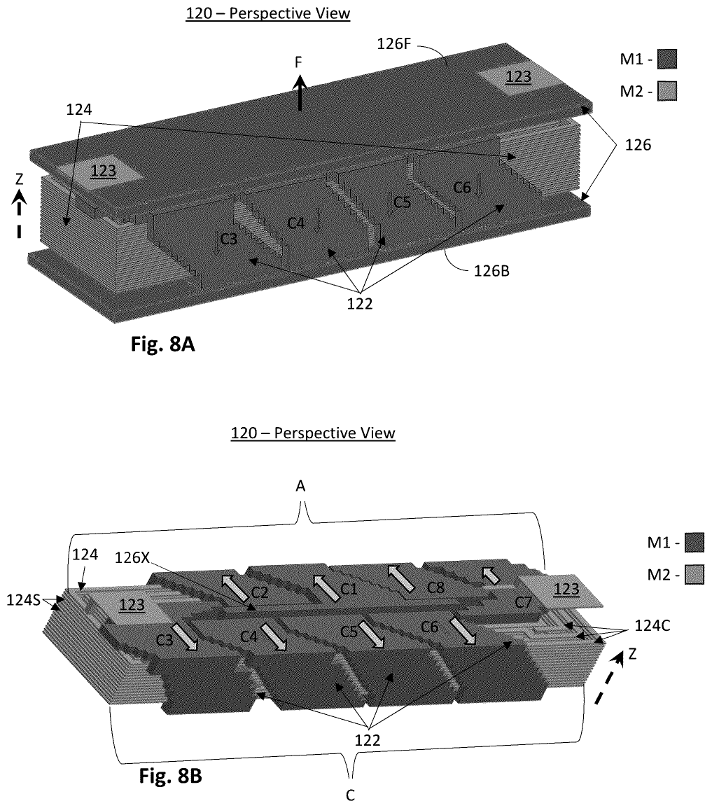

As indicated above, in certain embodiments of the present invention the magnetic channel 129 has a tapered shape/structure in a region thereof between the flux collection surfaces/facets of the flux collectors 126 and the magnetic core sections 122. The example illustrated in FIGS. 6A to 6C exemplifies such tapered shape of the magnetic channel 129 in which the cross-section area/width of the magnetic channel 129 is getting narrower along the direction advancing from the flax collectors 126 towards the core sections 122 of the channel. In this non-limiting example, the tapering of the magnetic channel 129 is made gradually at several location including tapering of the flux collectors 126, as shown in the tapered regions 129A marked in FIG. 3A, as well as tapering of the section 129B of the magnetic channel 129 that directs the magnetic flux between the flux collectors 126 to the core sections 122 as illustrated in FIG. 6C. The tapering provides for improved coupling of external magnetic flux propagating collected by the flux collectors 126 to flow through the magnetic channel 126, and thereby improves the sensitivity of the coil 120. It should be noted that the tapering of the magnetic channel towards the magnetic core thereof, as exemplified in this embodiment in regions 129A and/or 129B, may also be implemented in other embodiments of the present invention, for example in coils 120 with configurations such as exemplified in FIGS. 1A to 1C, 3A to 5D described above and/or in FIGS. 7A to 8B described below.

FIGS. 7A and 7B exemplify, in self-explanatory manner, a yet another example of an electric component including a coil 120, which may be 3D according to the present invention. FIG. 7A depicts a perspective view of the coil 120 and FIG. 7B depicts a cross-sectional view of the coil 120 taken along the plane CP illustrated in FIG. 7A. In this example, the magnetic channel 129 extends between the flux collectors 126 in a meander-like shape/path. The flux collectors 126 are not shown in FIG. 7B, yet instead one of the regions 126X of the magnetic channel 129 is shown to which one of the flux collectors 126 is coupled. As in the example of FIGS. 4A and 4B, also in this example the magnetic channel 129 is printed with the curves/folds 127 such that core section(s) 122 thereof (in this case a plurality of core sections) extend substantially along the printing direction Z. Accordingly the magnetic core sections 122 are surrounded by turns of the conductive windings 124 which include a plurality of conductive turns surrounding different one or more of the magnetic core sections 122. To this end, in various embodiments, the electric channel 128, may be printed as a single conductive path passing between two electric contacts/terminals (not specifically shown) at opposite ends thereof, and shaped to form the winding sets 128A to 128D along a single conductive path, or the electric channel 128 between the electric contacts may be printed to split into several (e.g. 4) electric sub channels forming winding sets 128A to 128D which may be electrically connected to one another (e.g. in series in order to increase the output voltage of the coil).

The 3D printed coils 120 with the meander-like and/or helical-like magnetic channels 129, as exemplified in FIGS. 5A to 7B, may be advantageous in some cases in terms of their sensitivity. This is due to the shapes of their magnetic channel which facilitates packing longer total core sections 122 and thus higher count of windings' 124 turns surrounding them, within electric components/coils 120 having compact dimensions. In particular, coils 120 with the meander-like and/or helical-like magnetic channels 129 are suited for forming flattened coils similar to that illustrated in the embodiment of FIGS. 4A and 4B, and the features of the flattened coil 120 exemplified in FIGS. 4A and 4B may also be implemented in the embodiments of FIGS. 5A to 7B. Additionally, the windings 124, and/or the winding sets 128A to 128D in the embodiments of FIGS. 5A to 7B may in some embodiments be implemented with concentric winding configuration including a plurality of concentrically arranged turn stacks similar to the windings 124 illustrated in FIGS. 3A to 3I, in order to yield improved sensitivity of the coil 120. In this regard, in some implementations the lateral spacing between concentrically arranged turns may be of only one voxel in a manner similar to that described with reference to FIGS. 3A to 3I, in order to facilitate more compact packing of higher count of turns within compact dimensions thus further improving the coil's sensitivity.

With reference to FIGS. 8A and 8B illustrated is a 3D printed coil 120 (e.g. or of an electric component containing it) according to an embodiment of the invention, in which the magnetic channel 129 is curved/folded to follow/form a coiled-like path/shape (e.g. such that it is wrapped about the conductive windings 124 of the coil 120). FIG. 8A depicts a perspective view of the coil 120 including the flux collectors 126, and FIG. 5B depicts another perspective view of the coil 120 without showing the flax collectors 126, so that the internal configuration of the coiled magnetic core 122 and its wrapping about the conductive windings 124 is visible in the figure. Like reference numerals are used in FIGS. 8A and 8B to designate elements/sections/parts of similar functionality as in the coils of the other embodiments of the invention described above.

To this end, in this example the magnetic flux collected by one of the flux collectors 126 is channeled by the magnetic channel 129 to circumference the conductive turns of the windings 124, typically for a plurality of times. For instance, in this non-limiting example, magnetic flux collected from the top flux collection surface 126U illustrated in FIG. 8A is channeled through the magnetic channel 129 from the region 126X of the magnetic channel 129 (being the connection between the flux collector to the coiled magnetic core 122) to follow sequentially through coiled sections C1 to C8 of the core 122 and then exit the coil 120 though the bottom flux collection surface 126D. The direction of the flux flow through the coiled core sections C1 to C8 of the magnetic core 122L is depicted in self-explanatory manner. To this end, the coiled core sections C1 to C8 are wrapped/winded about the turns of the conductive windings 124 (e.g. intertwined therewith) in a manner that induces voltage/current in the conductive windings 124 in response to magnetic flux propagating through the coiled core sections C1 to C8, and thereby voltage at the electric contacts/terminals 123 of the electric channel 128.

It should be noted that the coiled core configuration illustrated in this embodiment is suitable for fabrication of a flattened coil, as described above, whereby the printing direction Z is not necessarily transverse to the magnetic axis F of the coil 120. This is because the coiled core configuration illustrated in this example, facilitates longer core sections 122 to be winded about the windings even when the distance between the flux collectors 126 is small. Indeed, as exemplified in the optional configuration of the coil 120 illustrated in FIGS. 8A and 8B, the printing direction Z is substantially parallel to the magnetic axis F. The winding turns are preferably 3D printed with planar orientations within the printing layers, as exemplified in these figures, however it should be noted that in other embodiments the winding turns may also be 3D printed in other orientations.

Alternatively, or additionally, although not illustrated in the figures, it should be understood and appreciated by those versed in the art that a coil 120 with similar coiled configuration of the magnetic core 122 may be fabricated such that its magnetic axis F is perpendicular to the printing direction Z, thus optionally implementing a flattened coil configuration similar to that described above with reference to FIGS. 4A and 4B but with a coiled configuration of the core 122. For instance, in a none-limiting example, such flattened coil configuration would be obtained by arranging the flux collectors 126 to be located from the lateral sides A and C of the coil 120 which are marked in FIG. 8B; or alternatively 3D printing the turns of the conductive windings 124 with orientation perpendicular to the magnetic axis (e.g. parallel to the printing direction, such the turns or some of them are printed across multiple printed layers).

It should be noted that in this non-limiting example, in order to provide further improved sensitivity of the coil 120, the conductive windings include plurality of turns 124S stacked along the printing direction Z in a plurality of printing layers (e.g. as indicated above in some embodiments printing the turns with such orientation facilitates the relatively small spacing between the stacked turns due to the layers' thicknesses being substantially smaller than the lateral width of the printed voxels). Optionally in at least some of the printing layers the multiple turns 124C are printed concentrically in order to provide further improved sensitivity of the coil 120. To this end in some implementations the turns of the windings 124 are printed with similar configuration and insulation between them as illustrated in the example of FIGS. 3A to 3I to thereby obtain compact packing of a plurality of conductive turns surrounding the core sections C1 to C8, thus yielding highly sensitive coil 120 having compact dimensions.

To this end, the figures described above, schematically illustrate various non-limiting examples of 3D printed electric components 100, and/or of 3D printed coils 120. Preferably, in some implementations the turns of the conductive windings 124 of the coils are 3D printed planarly within the printing layers in order to exploit the smaller pitch between printing layers, which is smaller than lateral dimensions of the printed voxels, for packing/printing a higher count of conductive winding turns in the coils and facilitate high sensitivity of coils (i.e. facilitate high induced voltage in response to magnetic field sensing). Additionally, or alternatively, in some implementations, functional features of the coils/electric-components are 3D printed with minimal in-layer feature size of at least two voxels, in order to yield reliable 3D printing of the coils/electric-components. Additionally, in some implementations concentric winding turns are packed compactly with lateral spacing of only one voxel between concentrically adjacent turns. The invention also provides a technique for fabrication a high sensitivity flattened coil having a low/small aspect ratio between its length along its magnetic axis and its width traverse to its magnetic axis. This may be achieved by 3D printing at opposite ends of the magnetic channel 129, a pair of flux collectors 126 facing the magnetic axis F to collect flux propagating therefrom, and further printing the magnetic channel 129 with turn(s)/fold(s)/curve(s) 127 between the pair of flux collectors so that one or more core sections 122 thereof extends along the printing direction Z and passes through the conductive windings 124. Alternatively, or additionally, as indicated above a highly sensitive flattened coil may also be fabricated by utilizing a coiled core configuration. It should be understood that although the above features of the present invention are in some cases exemplified in different figures for clarity, various combinations of these features which may not have been specifically illustrated in the figure, may be implemented in coils/electric components fabricated according to the invention while without departing from the present invention.

Furthermore, it should be noted that some optional properties of the 3D printed coils, which were illustrated only in some embodiments described above, can be readily implemented in other configurations of coils 120 of the invention without departing from the invention. For instance, as would be appreciated by those versed in the art, the following optional properties/features may be implemented in various embodiments of the invention and not only in those they were depicted above:
the splitting of the magnetic channel 129 to form a plurality of magnetic subchannels thereof between the flux collectors as illustrated in FIGS. 6A to 6C;
the splitting of the electric channel to form multiple conductive paths and optionally multiple winding sets, as illustrated in FIGS. 3A to 3I and 6A to 6C; and
the formation of a conductive windings with both stacking of turns in multiple layers as well as forming concentric turns in each, or at least some of the layers as illustrated in FIGS. 3A to 3I and 8A to 8B.

With reference back to the method 200, as indicated in FIG. 2 optionally in some embodiments method 200 further includes operation 250 for fabricating/attaching surface mount contacts to the 3D printed electric components 100 or to the coil(s) 120, to facilitate their installation on a circuit board via surface mount technology (SMT).

According to some embodiments of the invention, surface mounts suitable for the SMT are 3D-printed together with the 3D-printing of the components 100. In such embodiments typically materials such as silver-palladium alloy are used for the SMT mounts' electric contacts, since the conventional materials, Tin and Nickel-Tin which are typically used in SMT mounts' electric contacts, are not suitable for the sintering process of the 3D printing technology of the invention (Tin's melting point is too low and Nickel-Tin will oxidize in the process). To this end in some embodiments the 3D-printing operation 220 of method 200 further includes 3D printing of the external/SMT contacts at respective ends of the electric contact/terminals 123 of the coils 120 or of the electric components 100. The 3D printed SMT contacts may be for example fabricated from silver-palladium alloy (Ag—Pd) and formed with thickness of about 9 μm or more to yield external Ag—Pd SMT electrodes for the 3D printed electric components 100.

Alternatively, or additionally, in some embodiments of the invention the SMT contacts/mounts, or some of them, comprise for example chemically/electrochemically deposited Tin-plated Copper or Nickel and are fabricated/attached to the 3D printed electric components 100/coils 120 and coupled to their electric contacts 123 only after the 3D-printing and Sintering of the components 100/coils 120.

Typically, one or more of the coils 120 of the sensor 100, or all of them, are designed with an open magnetic circuit configuration of their magnetic channel 129/core 122; that is suitable for accurate sensing of magnetic fields, and in particular suitable for magnetic field bases position sensing in medical applications/devices such as in catheters. In this context, the phrase open magnetic circuit configuration should be understood herein as relating to coil structures with an open-ended magnetic channel that poses substantially no shielding or shunting of external magnetic fields enabling the external magnetic field pass through the coil's magnetic core.

As indicated above the 3D printed magnetic field sensor 100 of the present invention as described above, particularly when printed with the coils 120 having open magnetic circuit configurations, may be suited for use as position and/or force sensors in medical instrument/device requiring the same.

Reference is now made to FIG. 9 illustrating a side-view of a medical instrument 1000 (e.g., probe/catheter) according to an embodiment of the present invention in which a position sensor 300 including 3D printed coils 120 configured according to the present invention, (and/or electric component(s) 100 of the invention including the same).

The medical instrument 1000 includes a housing H with a magnetic field-based position sensor 300 furnished therein. The medical instrument's housing H includes a main section M and a tip section T which is coupled at a distal end of the main section M via a banding coupler J (e.g. a spring or joint). The banding coupler J is configured such that the orientation of the tip section T can be bent relative to the longitudinal axis LX of the housing H under applied force FC (which may be applied for example when tip section T of the medical instrument 1000 is in contact with a body tissue of a patient) with banding degree corresponding to a magnitude and direction of the applied force FC.

In this specific non-limiting example, the position sensor 300 a location and orientation sensor 320, and a force sensor 310, which are implemented by 5 coils 120.1 to 120.5 one or more of which are configured and operable according to the present invention.

The location and orientation sensor 320 utilizes 3 of the coils, including coils 120.4, 120.5 and at least one of the coils 120.1 to 120.3 for measuring magnetic fields produced by one or more magnetic field sources MFL that are located externally to the instrument 1000, and which produce magnetic fields whose measurements are indicative of the spatial location and orientation of the medical instrument 1000. The coils 120.4, 120.5 and the at least one coil of the coils 120.1 to 120.3 are arranged such that their magnetic axes span 3 dimensions to thereby facilitate determining the 3D location and/or orientation of the medical instrument 1000 based on measurements of the magnetic fields form the magnetic field sources MFL by said coils.

The force sensor 310 utilizes the 3 coils 120.1 to 120.3 in order to determine the relative of orientation between the tip and the main sections, T and M, of the housing H. As indicated above the relative of orientation between the tip and the main sections, T and M, is indicative of the force FC applied by the tip section T, for example on a patient's tissue. To this end the force sensor 310 also includes a magnetic field source MF_{O} that produces a reference magnetic field whose measurement by the coils 120.1 to 120.3 provide indication of the relative orientation between the tip and main sections, T and M. The coils 120.1 to 120.3 of the orientation sensor 310 and the magnetic field source MF_{O} are arranged in the housing H at different sides of the joint J, so that the measurements of the magnetic field from the magnetic field source MF_{O} by the coils of the orientation sensor 310 provide indication of the relative orientation between the tip and the main sections, T and M. For instance, typically, the magnetic field source MF_{O} is arranged at the tip section T and the coils 120.1 to 120.3 of the orientation sensor 310 are arranged at the main section M with their magnetic axes parallel to the longitudinal axis LX of the housing H (although opposite arrangement may also be applicable). To this end the coils 120.1 to 120.3 are generally arranged such that their magnetic axes are collinear to one another (while not being co-planar in order to facilitate the relative orientation to be measured with respect to two angles, e.g. indicative of the pitch and yaw of the tip T relative to the main section).

The specific details of the technique for magnetic-field based position sensing, which is used in the present example for sensing the location and orientation of the medical instrument 1000 and/or the forced applied thereby on a patient's tissue, are generally known in the art and need not be further discussed herein in more details. For instance, U.S. patent No. U.S. Pat. No. 8,535,308 and U.S. patent application No. U.S. 2020/015693, which are both assigned to the assignee of the present application, disclose example configurations of catheters utilizing sensory circuits having several coils, for sensing the location and orientation of the catheter body, as well as the force applied thereby at the interface with a tissue.

In the present nonlimiting example, the magnetic-field based position sensor 300 of the medical device 1000 is implemented with one or more of the coils 120.1 to 120.5 being a 3D printed open magnetic circuit coils, which are configured according the present invention and/or fabricated by the 3D printing method described above. In this example the housing has a narrow width/diameter of about 2 mm, and accordingly the coils 120.1 to 120.5 are compact coils extending typically no more than 0.5 mm in the radial direction with respect to the longitudinal axis of the medical instrument.

In the present non-limiting example, the three coils 120.1, 120.2 and 120.3 are 3D printed coils according to the present invention, which may be implemented as an integral part of a single electric component 100 which is 3D printed utilizing the method 200 of the present invention described above. The electric component 100 in this example has a cylindrical/donut structure with a hole along its symmetry axis in the middle, and is fitted within the housing H of the medical instrument such that its symmetry axis is parallel to the longitudinal axis LX (e.g. in order to enable passage of various connection/fluid lines of the medical instrument therethrough). The two coils 120.4 and 120.5 are also 3D printed coils according to the present invention, with SMT mounts fabricated/coupled thereto according to the optional method operation 250 described above. These coils 120.4 and 120.5 are furnished on a flexible circuit board CB which is folded/rolled to a cylindrical shape within the housing H of the medical instrument 1000, such that their magnetic axes are substantially not parallel to one another and also not parallel (typically perpendicular) to the longitudinal axis LX of the housing H. The two coils 120.4 and 120.5 are preferably similar coils having flattened geometry, and may optionally be configured according to any one of the embodiments of FIGS. 4A to 8B, which facilitate 3D printing of flattened coils with sufficient sensitivity for use in medical device position sensors. The conductive windings of any one or more of the coils 120.1 to 120.5 may also be implemented/configured according to the techniques described above with reference to FIGS. 3A to 3I to yield densely packed winding turns, and thereby improved sensitivity of the coils with compact dimensions. Also, preferably, the coils are fabricated such that their functional features are printed with minimal in-layer feature size of at least to voxels of the 3D printing, as described above with reference to FIGS. 1A to 1C.

It should be understood that although in the present non-limiting example all of the coils 120.1 to 120.5 used in the position sensor 300 are 3D printed coils of the present invention, in other embodiments of the invention some of the coils 120.1 to 120.5 are not necessarily be 3D printed coils, for instance coils 120.4 and 120.5 may be conventional wire winded coils, without departing from the invention. Moreover although in the present non-limiting example coils 120.1 to 120.3 are printed within a single electric component 100 and coils 120.4 and 120.5 are individually 3D printed coils furnished on a circuit board (in this non-limiting example furnished with SMT technology), in other embodiments of the present invention any number or combination of the coils 120.1 to 120.5 may be either 3D printed as individual coils and/or 3D printed as parts of one or more electric components containing them which are 3D printed according to the invention. For instance, in some implementations all the 5 coils may be part of a single 3D printed electric component according to the invention.

Advantageously, 3D printing of electric components and coils of the present invention facilitates reliable, accurate and cost-effective fabrication of open and fitting of open magnetic circuit coils within the narrow dimensions of small medical devices while facilitating sufficient sensitivity of the coils for serving as accurate position sensors of such medical device.

### EXAMPLES

Example 1. A method to fabricate an electric component including a coil device. The method includes 3D printing the coil device, by:
3D printing a magnetic material to form a magnetic channel including a magnetic core of the coil device;
3D printing a conductive material to form a conductive channel, such that the conductive channel includes conductive windings of the coil device with turns surrounding the magnetic core; and
3D printing a non-magnetic electrically insulating material to form electrical insulation between the turns of the conductive windings of the coil device;
wherein functional structures of the electric component, including the turns of the conductive windings of the coil device and the electrical insulation between them, are each 3D printed with minimal in-layer feature size of at least two voxels of the 3D printing, to thereby facilitate robust and reliable 3D printing of the electric component and mitigate voxel misprints.

Example 2. The method according to Example 1, wherein the 3D printing includes successive printing of a plurality of printed layers along a printing direction; and wherein the turns of the conductive windings are 3D printed planarly within the printed layers and are 3D printed in the multitude of the printed layers; and wherein the magnetic core passes through a consecutive multitude of the printed layers.

Example 3. The method according to Example 2, wherein at least some of the turns are 3D printed in consecutive layers.

Example 4. The method according to Example 2 or 3, wherein the method includes:
3D printing of electrical insulation between adjacent turns with minimal in-layer feature size of the electrical insulation of at least two voxels of 3D printed electrically insulating material; and
3D printing of conductive vias to electrically connect between turns in different layers.

Example 5. The method according to Example 4, wherein the turns of the conductive windings include adjacent concentric turns that are 3D-printed in consecutive layers of the 3D printed layers with lateral separation of one voxel between them (i.e. between the adjacent concentric turns).

Example 6. The method according to Example 5, wherein the lateral separation of the single voxel between adjacent concentric turns is arranged with respect to one or more lateral sides of the adjacent concentric turns, and the conductive vias are arranged along at least one other lateral side of the concentric turns; and wherein the lateral separation between the adjacent concentric turns along the at least one other lateral side is of at least two voxels in order to maintain electrical insulation with the minimal in-layer feature size of the at least two voxels along that at least one other lateral side.

Example 7. The method according to Example 6, wherein the turns include neighboring concentric turns 3D printed within the same layer and at least one adjacent concentric turn printed in a consecutive layer above or below the said same layer; and wherein a lateral separation between the neighboring concentric turns within the said same layer is of four voxels along the one or more lateral sides, and of six voxels along the at least one other lateral side; thereby facilitating the minimal in-layer feature size of the electrical insulation between each of the neighboring concentric turns and the at least one adjacent concentric turn.

Example 8. The method according to any one of Examples 1 to 7, wherein the magnetic channel is configured with an open magnetic circuit configuration, and the 3D printing of the magnetic channel includes printing a pair of flux collectors at opposite ends of the magnetic core having respective flux collection facets defining a magnetic axis of the coil between them.

Example 9. The method according to Example 8, wherein the flux collection facets of the flux collectors are wider than the magnetic core.

Example 10. The method according to Example 8 or 9, wherein the flux collectors are tapered with the narrower facets of their tapering connected at respective opposite ends of the magnetic core and their wider facets serving as the flux collection facets.

Example 11. The method according to any one of Examples 1 to 10, wherein the magnetic core has a rod-like shape.

Example 12. The method according to any one of Examples 8 to 11, wherein the coil is a flattened coil having a small/low aspect ratio between its length along its magnetic axis and its width traverse to its magnetic axis; and wherein the magnetic channel is 3D printed along a curved (e.g. folded) path such that one or more sections of the magnetic core extend along a transvers direction with respect to the magnetic axis of the coils with a length of the one or more sections (e.g. the total length thereof) being larger than a distance between the pair of flux collectors; and wherein the arrangement of conductive windings of the coil includes a plurality of turns surrounding the one or more sections of the magnetic core; thereby enabling to furnish high count of the turns of the conductive windings along the length of the one or more sections of the magnetic core and obtaining improved sensitivity of the coil.

Example 13. The method according to Example 12, wherein the curved path of the magnetic channel is such that the magnetic core has a straight, rod-like shape, extending along the transvers direction with respect to the magnetic axis.

Example 14. The method according to Example 12, wherein the curved path of the magnetic channel is such that the magnetic core has a helical-like shape or meander-like shape, and wherein the turns of the conductive windings surround one or more of said sections of the helical-like shaped or meander-like shaped magnetic core.

Example 15. The method according to Example 12, wherein the curved path of the magnetic channel is such that the magnetic core has a coiled shape surrounding one or more of the turns of the conductive windings.

Example 16. The method according to any one of Examples 1 to 15, wherein the method includes 3D printing a body of the electric component with non-magnetic electrically insulating material, at regions not occupied by the magnetic and conductive channels; and wherein the conductive material of the conductive channel is fully embedded within the body so as to be isolated from environmental conditions by which it might be degraded.

Example 17. The method according to any one of Examples 1 to 16, wherein at least one of the following:
the conductive material includes Silver;
the conductive material includes Copper;
the conductive material includes Palladium;
the conductive material includes Silver-Palladium alloy;
the magnetic material includes Ceramic-Ferrite material;
the magnetic material includes Metal Material;
the magnetic material is a soft magnetic material having relative permeability μᵣ in the order of 100 or more; and
the non-magnetic dielectric material includes ceramic or glass-ceramic material.

Example 18. The method according to any one of Examples 1 to 17, wherein the 3D printing comprises:
(a) providing curable resins including:
   conductive material resin including particles of conductive material suspended in curable polymer binder;
   non-magnetic dielectric material resin including particles of non-magnetic dielectric material suspended in curable polymer binder; and
(b) 3D printing the curable resins, wherein the 3D printing includes:
   3D printing and curing the magnetic material resin at regions of the magnetic channel;
   3D printing and curing the conductive material resin at regions of the conductive channel; and
   3D printing and curing the non-magnetic dielectric material regions of the electric component, which are not occupied by the magnetic and conductive channels;
   thereby forming a 3D printed structure of the electric component; and
(c) sintering the 3D printed structure of the electric component at temperature sufficient for burning, or driving off, polymers therefrom and thereby achieving ceramic or metal density approaching 100% in the electric component.

Example 19. An electric component fabricated by the method of any one of Examples 1 to 18.

Example 20. A flattened coil having small/low aspect ratio between its length along its magnetic axis and its width traverse to its magnetic axis; the coil includes:
a body having a bulk being 3D-printed with non-magnetic electrically insulating material;
a magnetic channel 3D printed within the body with magnetic material, wherein the magnetic channel includes: at least a pair of flux collectors having respective flux collection facets perpendicular to the magnetic axis of the coil, and a magnetic core of the coil connected between the at least two flux collectors; and
a conductive channel 3D printed in said body with electrically conductive material, wherein the conductive channel includes an arrangement of conductive windings with a plurality of turns surrounding the magnetic core; and
wherein the magnetic channel is 3D-printed with curved (e.g. folded) path such that one or more sections of the magnetic core extend along a transverse direction with respect to the magnetic axis of the coil, and the conductive channel is 3D-printed such that the turns thereof surround one or more of the sections of the magnetic core.

Example 21. An electronic component including at least one coil; the electronic component includes:
a magnetic channel including a magnetic core of the at least one coil, 3D-printed with magnetic material; and
a conductive channel 3D-printed with electrically conductive material, wherein the conductive channel includes an arrangement of conductive windings having a plurality of turns surrounding the magnetic core;
electrical insulation 3D-printed with non-magnetic electrically insulating material between the turns;
wherein 3D-printed functional structures of the coil, including the turns of the conductive windings and the electrical insulation between them, are each printed with a minimal in-layer feature size of at least two voxels of the 3D printing.

Example 22. The electronic component according to Example 21, being 3D printed by successive printing of a plurality of printed layers along a printing direction; and wherein each of the turns of the conductive windings is 3D-printed planarly within one of the printed layers and the turns are 3D printed in the multitude of the printed layers; and wherein the magnetic core passes through a consecutive multitude of the printed layers.

Example 23. The electronic component according to Example 22, wherein the electronic component includes electrical insulation 3D-printed between adjacent turns of the conductive windings with minimal in-layer feature size of the electrical insulation of at least two voxels of 3D-printed electrically insulating material; and the conductive channel includes conductive vias that electrically connect between turns in different layers.

Example 24. The electronic component according to Example 23, wherein the turns include concentrically adjacent turns 3D-printed in consecutive layers of the 3D printed layers, with lateral separation of one/single voxel between them (i.e. between the concentrically adjacent turns).

Example 25. The electronic component according to Example 24, wherein the lateral separation of the one/single voxel between concentrically adjacent turns is from one or more lateral sides of the adjacent concentric turns, and wherein the conductive vias are 3D-printed along at least one other lateral side of the concentric turns; and wherein the lateral separation between the adjacent concentric turns along the at least one other lateral side is of at least two voxels in order to maintain the minimal in-layer feature size of the insulation such that it is of at least two voxels along that at least one other lateral side.

Example 26. A method to fabricate a coil device having small/low aspect ratio between its length along its magnetic axis and its width traverse to its magnetic axis;
the method includes fabricating the coil by 3D-printing including:
3D-printing of a magnetic channel including: at least a pair of flux collectors having respective flux collection facets perpendicular to the magnetic axis, and a magnetic core of the coil having a magnetic channel connected between the at least two flux collectors; and
3D-printing a conductive channel including an arrangement of conductive windings with a plurality of turns surrounding the magnetic core;
wherein the magnetic channel is 3D printed with curved (e.g. folded) path such that one or more sections of the magnetic core thereof extend along a transverse direction with respect to the magnetic axis of the coil and with the turns of the conductive windings surround one or more of said sections.

Example 27. A coil device fabricated by the method of Example 26.

Example 28. A method to fabricate a coil device, the method includes:
3D-printing a magnetic material to form a magnetic channel including a magnetic core of the coil;
3D printing a conductive material to form a conductive channel including an arrangement of conductive windings with a plurality of turns surrounding the magnetic core; and
3D-printing a non-magnetic electrically insulating material to form electrical insulation between the turns;
wherein 3D-printed functional structures of the coil, including the turns and the electrical insulation, are each printed with minimal in-layer feature size of at least two voxels of the 3D-printing;
wherein the turns comprise concentrically adjacent turns 3D-printed in consecutive 3D-printed layers of the 3D-printing with lateral separation of one/single voxel between the concentrically adjacent turns.

Example 29. The method according to Example 28, wherein the lateral separation of the one/single voxel is 3D-printed relative to one or more lateral sides of the concentrically adjacent turns; wherein the 3D-printing of the conductive channel includes 3D printing of conductive vias along at least one other lateral side of the turns for electrically connecting between turns in different layers; and wherein the lateral separation between the adjacent concentric turns along the at least one other lateral side is of at least two voxels in order to maintain electrical insulation between the turns with minimal in-layer feature size at least two voxels along the at least one other lateral side.

Example 30. A coil device fabricated by the method of Examples 28 or 29.

1. A method to fabricate an electric component including a coil device, the method comprising
3D printing the coil device; wherein said 3D printing comprises:
3D printing a magnetic material to form a magnetic channel comprising a magnetic core of the coil device;
3D printing a conductive material to form a conductive channel, said conductive channel comprising conductive windings of the coil device with turns surrounding the magnetic core; and
3D printing a non-magnetic electrically insulating material to form electrical insulation between the turns of the conductive windings of the coil device;
wherein functional structures of the electric component, including said turns of the conductive windings of the coil device and said electrical insulation between them, are each printed with minimal in-layer feature size of at least two voxels of said 3D printing, to thereby facilitate robust and reliable 3D printing of the electric component and mitigate voxel misprints.

2. The method according to claim 1, wherein said 3D printing comprises successive printing of a plurality of printed layers along a printing direction; and wherein said turns of the conductive windings are 3D printed planarly within said printed layers and are 3D printed in the multitude of the printed layers; and wherein said magnetic core passes through a consecutive multitude of said printed layers.

3. The method according to claim 2, wherein at least some of said turns are 3D printed in consecutive layers.

4. The method according to claim 2 comprising:
3D printing of electrical insulation between adjacent turns with minimal in-layer feature size of the electrical insulation of at least two voxels of 3D printed electrically insulating material; and
3D printing of conductive vias to electrically connect between turns in different layers.

5. The method according to claim 4, wherein said turns comprise adjacent concentric turns printed in consecutive layers of said 3D printed layers with lateral separation of one voxel between them.

6. The method according to claim 5, wherein said lateral separation of the one voxel between adjacent concentric turns is arranged with respect to one or more lateral sides of the adjacent concentric turns, and wherein said conductive vias are arranged along at least one other lateral side of the concentric turns; and wherein the lateral separation between the adjacent concentric turns along said at least one other lateral side is of at least two voxels in order to maintain electrical insulation with said minimal in-layer feature size of the at least two voxels along said at least one other lateral side.

7. The method according to claim 6, wherein said turns comprise neighboring concentric turns 3D printed within the same layer and at least one adjacent concentric turn printed in a consecutive layer above or below said same layer; and wherein a lateral separation between said neighboring concentric turns in said same layer is of four voxels along said one or more lateral sides, and of six voxels along said at least one other lateral side; thereby facilitating said minimal in-layer feature size of the electrical insulation between each of said neighboring concentric turns and said at least one adjacent concentric turn.

8. The method according to claim 1, wherein said magnetic channel is configured with an open magnetic circuit configuration, and said printing of the magnetic channel comprises printing a pair of flux collectors at opposite ends of said magnetic core having respective flux collection facets defining a magnetic axis of the coil between them.

9. The method according to claim 8, wherein said flux collection facets of the flux collectors are wider than said magnetic core.

10. The method according to claim 8, wherein the flux collectors are tapered with the narrower facets of their tapering connected at respective opposite ends of the magnetic core and their wider facets serving as said flux collection facets.

11. The method according to claim 1, wherein said magnetic core has a rod-like shape.

12. The method according to claim 8, wherein said coil is a flattened coil having a small aspect ratio between its length along its magnetic axis and its width traverse to its magnetic axis; and
wherein said magnetic channel is 3D printed along a curved path such that one or more sections of the magnetic core extend along a transvers direction with respect to the magnetic axis of the coils with a length of said one or more sections being larger than a distance between said pair of flux collectors; and wherein said arrangement of conductive windings includes a plurality of turns surrounding said one or more sections of the magnetic core;
thereby enabling to furnish high count of said turns along the length of the one or more sections of the magnetic core and obtaining improved sensitivity of said coil;

13. The method according to claim 12, wherein said curved path of the magnetic channel is such that the magnetic core has a straight, rod-like shape, extending along the transvers direction with respect to the magnetic axis.

14. The method according to claim 12, wherein said curved path of the magnetic channel is such that the magnetic core has a helical-like shape or meander-like shape, and wherein said turns of the conductive windings surround one or more of said sections in the helical-like shaped or meander-like shaped magnetic core.

15. The method according to claim 12, wherein said curved path of the magnetic channel is such that the magnetic core has a coiled shape surrounding one or more of said turns of the conductive windings.

16. The method of claim 1, wherein said 3D printing comprises:
(a) providing curable resins comprising:
magnetic material resin comprising particles of magnetic material suspended in curable polymer binder;
conductive material resin comprising particles of conductive material suspended in curable polymer binder;
non-magnetic dielectric material resin comprising particles of non-magnetic dielectric material suspended in curable polymer binder; and
(b) 3D printing said curable resins comprising:
3D printing and curing said magnetic material resin at regions of said magnetic channel;
3D printing and curing said conductive material resin at regions of said conductive channel; and
3D printing and curing said non-magnetic dielectric material regions of said electric component not occupied by said magnetic and conductive channels;
thereby forming a 3D printed structure of the electric component; and
(c) sintering said 3D printed structure of the electric component at temperature sufficient for burning or driving off polymers therefrom and thereby achieving ceramic or metal density approaching 100% in said electric component.

17. A coil having a small aspect ratio between its length along its magnetic axis and its width traverse to its magnetic axis; the coil comprising:
a body having a bulk 3D printed with non-magnetic electrically insulating material;
a magnetic channel 3D printed in said body with magnetic material, wherein the magnetic channel comprises: at least a pair of flux collectors having respective flux collection facets perpendicular to said magnetic axis of the coil, and a magnetic core of the coil connected between the at least two flux collectors; and
a conductive channel 3D printed in said body with electrically conductive material, wherein the conductive channel comprises an arrangement of conductive windings with a plurality of turns surrounding the magnetic core;
wherein the magnetic channel is 3D printed with curved path such that one or more sections of the magnetic core extend along a transverse direction with respect to the magnetic axis of the coil and wherein the conductive channel is 3D printed such that said turns of the conductive windings surround one or more of said sections of the magnetic core.

18. An electronic component comprising at least one coil; the electronic component comprising:
a magnetic channel comprising a magnetic core of the at least one coil, 3D printed with magnetic material; and
a conductive channel 3D printed with electrically conductive material, wherein the conductive channel comprises an arrangement of conductive windings with a plurality of turns surrounding the magnetic core;
electrical insulation 3D printed with non-magnetic electrically insulating material between the turns;
wherein 3D printed functional structures of the coil, including said turns and the electrical insulation between them, are each printed with minimal in-layer feature size of at least two voxels of the 3D printing.

19. The electronic component according to claim 18, being 3D printed by successive printing of a plurality of printed layers along a printing direction; and wherein each of said turns of the conductive windings is 3D printed planarly within one of said printed layers and said turns are 3D printed in the multitude of the printed layers; and wherein said magnetic core passes through a consecutive multitude of said printed layers.

20. The electronic component according to claim 19 comprising electrical insulation 3D printed between adjacent turns of said conductive windings with minimal in-layer feature size of the electrical insulation of at least two voxels of 3D printed electrically insulating material; and said conductive channel comprising conductive vias 3D printed to electrically connect between turns in different layers.

21. The electronic component according to claim 20, wherein said turns comprise concentrically adjacent turns 3D printed in consecutive layers of said 3D printed layers with lateral separation of one voxel between them.

22. The electronic component according to claim 21, wherein said lateral separation of the one voxel between concentrically adjacent turns is from one or more lateral sides of the adjacent concentric turns, and wherein said conductive vias are 3D printed along at least one other lateral side of the concentric turns; and wherein the lateral separation between the adjacent concentric turns along said at least one other lateral side is of at least two voxels in order to maintain said minimal in-layer feature size of the insulation to be at least two voxels along said at least one other lateral side.
